(19) United States
(12) Patent Application Publication
(10) Pub. No.: US 2019/0274914 A1
(43) Pub. Date: Sep. 12, 2019

(54) DEVICES AND SYSTEMS FOR MANIPULATING TISSUE AND METHODS FOR USING SAME

(71) Applicants: Jana L. Hunter, Atlanta, GA (US); William Jacob Thompson, Atlanta, GA (US); Harrison Tyler Fox, Atlanta, GA (US); Mikaela Marie Leeyi Juzswik, Atlanta, GA (US); Timothy Edward Zollers, Atlanta, GA (US); Luke John Pfost, Atlanta, GA (US)

(72) Inventors: Jana L. Hunter, Atlanta, GA (US); William Jacob Thompson, Atlanta, GA (US); Harrison Tyler Fox, Atlanta, GA (US); Mikaela Marie Leeyi Juzswik, Atlanta, GA (US); Timothy Edward Zollers, Atlanta, GA (US); Luke John Pfost, Atlanta, GA (US)

(21) Appl. No.: 16/295,422

(22) Filed: Mar. 7, 2019

Related U.S. Application Data
(60) Provisional application No. 62/639,563, filed on Mar. 7, 2018.

(51) Int. Cl. A61H 7/00; A61F 7/00; A61H 23/00

(52) U.S. Cl. CPC A61H 7/004; A61F 7/007; A61H 23/004; A61F 2007/0071; A61F 2007/0075; A61H 2201/0228; A61H 2201/501; A61H 2201/0221; A61H 2201/1669; A61H 2201/0285; A61H 2201/1676

(57) ABSTRACT

Disclosed herein are devices and systems for stimulating and manipulating tissue, such as skin, fascia and muscle tissue, to reduce pain or discomfort. In one aspect, a tissue stimulation and manipulation device includes a manipulating head having a casing with an application area configured to contact at least one site of a subject; and a propulsion source capable of producing mechanical motion or linear energy; the propulsion source and application area are operatively positioned relative to one another such that the propulsion source and contact portions on the application area cooperate to manipulate tissue at the site of the subject. Also disclosed herein are methods for using the disclosed devices and systems to treat skin, fascia and muscle tissue.

### CROSS REFERENCE TO RELATED APPLICATIONS

This application claims the benefit of priority to U.S. provisional patent application Ser. No. 62/639,563 filed Mar. 7, 2018, which is herein incorporated in its entirety.

### FIELD OF INVENTION

The present invention relates to methods, devices, and systems for stimulating and manipulating tissue, such as skin, fascia and muscle tissue.

### BACKGROUND OF THE INVENTION

Healthy skin, fascia, and muscle restoration can lead to pain reduction, improved flexibility in joints, increase circulation and ultimately smooth and tighten skin. It may also drastically decrease the appearance of cellulite and helps the body restore to its healthiest muscle state. Current devices for treating fascia are manual, self-administered tools that are extremely labor intensive to utilize, particularly at the level needed for maximum results on a self-administered basis. Accordingly, there remains a need for new devices and systems which are automated, capable of hands-free operation and have the ability to be adjusted in a multitude of positions in order to access a wide variety of both front and back body parts. This need and other needs are satisfied by the various aspects of the present disclosure.

### SUMMARY OF THE INVENTION

In accordance with the purposes of the invention, as embodied and broadly described herein, the invention, in one aspect, relates to devices and systems for stimulating and manipulating tissue, such as skin, fascia and muscle tissue.

In another aspect, the invention relates to a device for stimulating and manipulating tissue at a site in a subject, the device comprising: a manipulating head with a casing comprising an application area, the application area including one or more portions configured to contact at least one site of the subject; and a pressure or propulsion source capable of exerting a force; wherein the propulsion source and contact portions are operatively positioned relative to one another such that the propulsion source and contact portions cooperate to manipulate tissue at the site of the subject. When a user activates the device, the application area of the casing may be configured to exert forces onto the user's body to press on, stimulate, or otherwise manipulate the underlying tissue.

In another exemplary aspect, the invention may allow a subject or user to adjust the mechanical manipulation operating parameters by using a control unit. For example, the user may use a wireless device to control speed and or force of the manipulation to low or first level, a medium or second level, or a high or third level. Other embodiments may allow for only two levels of force, more levels of force, and/or a continuous scale of available force rather than discrete level(s). In yet another exemplary aspect, the invention relates to a system for stimulating and manipulating tissue at a site in a subject, the system comprising: a device comprising: a casing comprising an application area, the application area including one or more portions configured to contact at least one site of the subject; a pressure or propulsion source capable of exerting a force; and a thermal source capable of producing heat and cold; wherein the propulsion source and contact portions are operatively positioned relative to one another such that the propulsion source and contact portions cooperate to manipulate tissue at the site of the subject; a frame assembly comprising a frame member having first and second opposed sides and a base connected to at least one end of the frame member; wherein the frame assembly is configured to detachably connect or attach with the casing; a controller communicatively connected to the device and configured to control operation of the one or more device components based on instructions regarding operation of the device; and a control unit communicatively connected to the controller and configured to transmit instructions regarding the operation of the device to the controller; wherein the propulsion source is configured to move the casing in a predefined manner while the application area of the casing is in contact with the subject to thereby manipulate the tissue.

In another exemplary aspect, the invention relates to a method for manipulating tissue in a subject to treat muscle and fascia tissue. In still another exemplary aspect, a method of the present invention comprises reducing the pain or irritating sensation, comprising contacting a device of the present invention at the site where the pain or irritating sensation is initiated, initiating reciprocating mechanical manipulation by the device in an intermittent or continuous manner, optionally applying cold or heat simultaneously with the reciprocating mechanical manipulation, manipulating the tissue or fascia for a sufficient time to interfere with nerve transmission and/or loosen and/or release fascia tissue. In yet another exemplary aspect, a method of the present invention comprises reducing discomfort in an animal, comprising contacting a device of the present invention to a site of discomfort, initiating reciprocating mechanical manipulation by the device, optionally applying a thermal effect simultaneously with the reciprocating mechanical manipulation, and reducing the sensation of discomfort. In still another exemplary aspect, a method disclosed herein comprises treating one or more injured sites in a subject, such as for treating an injured site or area by providing wound healing and/or tissue repair, comprising contacting a manipulating head of a device disclosed herein on or adjacent to an injured site of a subject, such as a chronic wound, a burn, an acute or overuse injury, myofascial injury or inflammation; initiating reciprocating mechanical manipulation by the device intermittently or continuously (which provides stimulation to the application site), optionally applying cold or heat simultaneously with the reciprocating mechanical manipulation, manipulating for a predetermined time, and optionally moving the device to another injured site or to a second location adjacent to the first injured site. For example, for an acute or overuse injury, a device may be applied directly to the injury site. For example, at sites wherein skin integrity is disrupted, a method may comprise placing the manipulating head adjacent to the disrupted site. At sites where the skin integrity is not disrupted, a method may comprise placing the manipulating head directly on the site. In yet another exemplary aspect, a method disclosed herein comprises accelerating healing of one or more injured sites in a subject, such as for wound healing and tissue repair, comprising contacting a device disclosed herein on or adjacent to an injured site of a subject, such as an acute or chronic injury or chronic wound or a burn; initiating reciprocating mechanical manipulation by the device in an intermittent or continuous manner, optionally applying cold or heat simultaneously with the reciprocating mechanical manipulation, manipulating for a predetermined time, and optionally moving the device to another injured site or to another location adjacent to the first injured site.

In further aspects, various methods disclosed herein may be effective for both reducing pain and enhancing wound healing or repair at injured sites. By way of non-limiting example, by enhancing wound healing, there is generally a reduction in pain at that site. For example, by treating myofascial injury, myofascial pain is reduced. Methods of enhancing wound or tissue healing also comprise methods for reducing pain at an injured site. Methods of the present disclosure comprise methods for reducing pain associated with wounds or injured sites by using a device disclosed herein to apply reciprocating mechanical manipulation near, adjacent to or on a wound or injured site, initiating thermal effects to relieve pain associated with the wound or injured site. In still further aspects, the invention also relates to additional methods for using the disclosed devices and systems.

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several aspects of the invention and together with the description, serve to explain the principles of the invention.

FIG. 1 shows a depiction of a system including tissue manipulation device and frame assembly with a human model in accordance with an exemplary embodiment of the present invention.

FIG. 2 shows a depiction of a system including tissue manipulation device and frame assembly in accordance with an exemplary embodiment of the present invention.

FIG. 3 shows a depiction of a system including tissue manipulation device and frame assembly in accordance with an exemplary embodiment of the present invention.

FIG. 4 shows a depiction of a tissue manipulation device adjustably connected to frame member in accordance with an exemplary embodiment of the present invention.

FIG. 5 shows a depiction of a tissue manipulation device adjustably connected to frame member in accordance with an exemplary embodiment of the present invention.

FIGS. 6A-6B show depictions of a propulsion source used in connection with the tissue manipulation device in accordance with an exemplary embodiment of the present invention.

FIG. 7 shows a depiction of a propulsion source used in connection with the tissue manipulation device in accordance with an exemplary embodiment of the present invention.

FIG. 8 shows a depiction of a propulsion source used in connection with the tissue manipulation device in accordance with an exemplary embodiment of the present invention.

FIG. 9 shows a depiction of a detachable manipulating head connected to a propulsion source in accordance with an exemplary embodiment of the present invention.

FIG. 10 shows a depiction of a detachable manipulating head connected to a propulsion source in accordance with an exemplary embodiment of the present invention.

FIGS. 11A-11C show depictions of a manipulating head for use in connection with the disclosed tissue manipulation devices in accordance with an exemplary embodiment of the present invention.

FIGS. 12A-12D show depictions of a blading head attachment for use in connection with the disclosed tissue manipulation devices in accordance with an exemplary embodiment of the present invention.

FIGS. 13A-13D show depictions manipulating heads for use in connection with the disclosed tissue manipulation devices in accordance with exemplary embodiments of the present invention.

FIGS. 14A-14D show depictions of manipulating heads for use in connection with the disclosed tissue manipulation devices in accordance with exemplary embodiments of the present invention.

FIG. 15 show a depiction of a manipulating head including a heating source for use in connection with the disclosed tissue manipulation devices in accordance with an exemplary embodiment of the present invention.

FIG. 16 shows a method for using the disclosed tissue manipulation devices and systems in accordance with another exemplary embodiment of the present invention.

FIG. 17 shows a diagram of a system including a computing device for enabling operation of the disclosed devices and systems in accordance with another exemplary embodiment of the present invention.

### DETAILED DESCRIPTION OF THE INVENTION

The present invention can be understood more readily by reference to the following detailed description of the invention and the Examples included therein.

Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

All publications mentioned herein are incorporated herein by reference to disclose and describe the methods and/or materials in connection with which the publications are cited.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of” Unless defined otherwise, all technical and scientific terms used herein have the same meaning as commonly understood by one of ordinary skill in the art to which this invention belongs. In this specification and in the claims, which follow, reference will be made to a number of terms which shall be defined herein.

As used in the specification and the appended claims, the singular forms “a,” “an” and “the” include plural referents unless the context clearly dictates otherwise. Thus, for example, reference to “an element” includes two or more elements.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

As used herein, the term “manipulation” or “manipulating” means pressing, kneading, rubbing, massaging, loosening, releasing, activating and/or stimulating the outer surface of the skin, underlying fascia and/or muscle tissue. In various aspects, manipulation may lead to increased blood flow to the area, which can help treat unhealthy skin, fascia and muscle tissue.

As used herein, the term “propulsion” means a mechanical force for driving an object, for example, pushing forward an object.

Disclosed are the components to be used to manufacture the disclosed devices and articles of the invention as well as the materials themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the invention. Thus, if there are a variety of additional steps that can be performed it is understood that each of these additional steps can be performed with any specific aspect or combination of aspects of the methods of the invention.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Tissue Manipulating Devices and Systems

As briefly described above, the present disclosure relates, in various aspects, to devices and systems for pressing, kneading, rubbing, activating, stimulating or otherwise manipulating tissue in a subject, such as fascia or muscle tissue.

In one aspect, the present disclosure provides a device for manipulating tissue at a site in a subject. The tissue manipulating device comprises an interchangeable, manipulating “head” attachment connected to a propulsion source capable of producing mechanical motion, for example, reciprocating linear motion. The manipulating head attachment may have a body or a casing with an application area, the application area including one or more contact portions configured to contact at least one site of a subject, for example a human. As will be described in more detail, the propulsion source and manipulating head attachment may be operatively positioned relative to one another such that the propulsion source and application area (i.e., the area of the head attachment in contact with the subject) cooperate to manipulate tissue at the site of the subject. To this end, the propulsion source may be configured to move the head attachment in a predefined manner, for example, in a reciprocating linear motion, while the application area is in contact with the subject to thereby manipulate the tissue.

In another aspect, the present disclosure provides a system for stimulating or manipulating tissue at a site in a subject, the system comprising a frame assembly adjustably connected with one or more tissue manipulation devices. In further aspects, a tissue manipulation device can comprise a head attachment with a casing comprising an application area, the application area including one or more contact portions configured to contact at least one site of the subject, the head attachment detachably connected with a propulsion source capable of producing a reciprocating mechanical force; and a thermal source or element capable of producing heat and/or cold. In further aspects, the propulsion source and head attachment are operatively positioned relative to one another such that the propulsion source and contact portions cooperate to manipulate tissue at the site of the subject. The system may include a controller communicatively connected to the tissue manipulating device and configured to control operation of the one or more device components, for example, based on instructions regarding operation of the device; and a control unit, which may be a switch mounted to the frame assembly or a wireless device, communicatively connected to the controller and configured to transmit instructions regarding the operation of the device to the controller.

Consistent with embodiments of the present disclosure, the invention is described as including one or more tissue manipulation devices. In further aspects, the tissue manipulation devices used in the systems need not be identical. For example, a user may have a system according to an embodiment of the invention wherein one of the tissue manipulation devices has stronger or more robust propulsion source than the other, no or differently sized thermal elements, differently positioned contact portions on the application area, etc. In further aspects, each tissue manipulation device may comprise at least one propulsion source and a plurality of interchangeable head attachments. In yet further aspects, the propulsion source is configured to produce a mechanical force upon activation. If the subject is in contact with the application area of the head attachment, the subject will feel the forces directly against the underlying tissue.

In various aspects, the present invention provides a frame assembly configured for use with the disclosed devices, for example, to detachably connect or attach with a device housing or mount. In one aspect, a frame assembly of the present invention can comprise a central frame member having first and second opposed sides and a base connected to at least one end of the central frame member or a plurality of side frame connected to the central frame member. The frame assembly may be configured to be adjustably connected with the propulsion source to allow the propulsion source to move along the central frame member. In further aspects, the frame assembly comprises at least one vertical frame member having first and second opposed sides and a base connected to at least one end of the frame member. In still further aspects, the frame assembly further comprising at least one handle member pivotably connected to a portion of the frame member. In yet further aspects, the frame assembly may comprise a plurality of handle members pivotably or foldably connected to a portion of the frame member. In yet further aspects, the handle member may be pivotably or foldably connected to a portion of the frame member at a perpendicular angle when in a use position and may be configured to fold at the connection point to a storage position where the handle members are substantially parallel to the frame member. In another aspect, the frame assembly may comprise a plurality of side frame connected to a vertical, central frame member. In some aspects, the frame assembly includes at least one connected left side frame and at least one connected right side frame. In other aspects, the frame assembly includes two or more left side frames and two or more right side frame. Each pair of side frames may be arranged in left pairs and right pairs.

In further aspects, the device or a device housing may be pivotably connected to the frame member, or other portions of the frame assembly using a connecting means. In still further aspects, the device and/or a device housing may be pivotably connected to the frame member, propulsion source, or other portion of the device using a joint. In yet further aspects, the joint may comprise a pivot ball with an arm extending therefrom, a socket for receiving said ball, and optionally a retaining means for securing the pivot ball in the socket. In still further aspects, the pivot ball arm may be connected to the casing and the socket for receiving said ball is connected to another portion of the device, such as the frame member or propulsion source; or vice versa. In various aspects, the device and/or device casing may be configured to rotate or swivel about an axis.

According to various further aspects of the invention, the frame assembly can be collapsible or foldable. In further aspects, the frame assembly is configured to collapse from an expanded state to a folded state, and expand from a folded state to an expanded state. In still further aspects, in the expanded state, the first and second left side frames are connected to the frame assembly, namely, the second ends of upper and lower second frame members may be hingedly connected to central frame member to allow left side frames collapse against one another and right side frame to collapse against one another. In yet further aspects, each pair of first and second side frames may be pivotably or hingedly connected to the central frame member of the frame assembly such that each pair of side frames are collapsible against one another. In further aspects, the frame assembly may comprise a locking mechanism configured to releasably secure the frame assembly in the expanded state and collapsed state. In the expanded state, the first and second side frames of each pair form a 90-degree angle or substantially 90-degree angle. In the collapsed state, the first and second side frames of each pair are parallel or substantially parallel to one another.

Frame members used to construct the frame assembly may be in the shape of a three-dimensional polygon, such as a tubular or cylinder shape, and the frame walls may define an interior space or interior sections for containing various operating elements of the invention as described herein. Any other shape (as used herein, the term shape is used in the broad sense of three-dimensional works) may be employed, so long as the shape is structured so as to be able support the methods of using the invention as more fully disclosed below. In some aspects, the frame can comprise a plurality of connected tubular members. In further aspects, the device may be comprised of any desired material, such as cast metal (e.g., aluminum), and preferably without seams, edges, or burrs on any part of the application area or members attached to the device casing. The device and system can be configured and constructed (e.g., using an angled frame member relative to the legs) to allow users to be able to lean into the device and frame assembly as needed to effectively receive treatment and without compromising the speed and distance settings, and stability.

In further aspects, the propulsion source may be configured to move the casing in a predefined manner while the application area of the manipulating head attachment casing is in contact with the subject to thereby manipulate the tissue. In still further aspects, the propulsion source can comprise any desired means for producing mechanical motion or force. In some aspects, the propulsion source may comprise at least one of: a motor, an actuator, screw drive, worm screw, barrel screw, wheel and axle, cam, rod, gear, track, belt, chain, rack and pinion, or the like. In other aspects, propulsion source may include reciprocating mechanism and/or drive mechanism including bearing guide rails, gear track and/or gear motor for generating reciprocating linear motion. The reciprocating mechanism may employ lower rail contact/upper gear engagement and upper rail contact/lower rack gear engagement. The reciprocating mechanism can allow for driving motor to spin one direction continuously to produce to back and forth motion using a circular rack with pinion gear system. In still further aspects, reciprocating mechanism uses a reciprocating motion with at least two inches of travel, for example, 3, 4, 5, 6, or more inches of travel.

In various aspects, a manipulating head attachment of the present invention can comprise a body or a casing having an application area. In further aspects, the application area may include one or more contact portions configured to contact at least one site of the subject. In further aspects, the casing may be configured to contain one or more components, for example a thermal and/or vibratory element. In some aspects, the casing may be shaped to conform to the contour of a surface of the body, may be configured to contain at least one vibratory element, and an optionally a thermal element. In other aspects, a casing or at least one surface of a casing, may be substantially planar to fit a flat surface of the body. In yet other aspects, a casing or at least one surface of a casing, may be shaped to fit a curved surface of the body. For example, one surface of a casing may be concave, shaped like the inner surface of a circle, and when the device contacts a surface, such as the top portion of a body, the concave surface of the casing substantially contacts the body surface, meaning that a majority of the concave surface is in contact with the area of the surface. This contact of substantially the entire concave surface of the device allows for enhanced transfer of vibration and/or thermal effect to the surface.

In various further aspects, contact portions of the head attachments may comprise raised surface portions of the application area and/or various protrusions, tips, or elements disposed on the application area. In still further aspects, the application area of the manipulating head attachment may have a specific configuration, arrangement, pattern, and/or surface profile. For example, the contacting portions may have a spacing to ensure equal pressure distribution between the contacting portion when applied to a subject's site. In yet further aspects, each of the raised portions and/or contacting surfaces may have a uniform shape and/or size. In further aspects, each of the raised portions have a varying shapes and/or sizes. In yet further aspects, the raised portions may have a semi-circular or concave shape. In even further aspects, the raised portions may have a sinusoidal shape or pattern. In still further aspects, the proximal ends of the raised portions (e.g., which contact the tissue of the subject) may be rounded or substantially planar. In even further aspects, the contact portions may further comprise one or more contact elements extending out therefrom, such as for applying pressure. In some aspects, the contact portions comprise a plurality of contact elements or tips.

In other aspects, the application area of the manipulating heads and/or attachments can be configured or shaped for use in “blading” or “scraping”. The application area may have a curved or blade shape and/or curves. However, the application area may be of any surface profile or skin contacting means that achieves the function of scraping and blading skin, fascia and/or muscle tissue of the subject. In some aspects, blading or scraping action may be understood to acts as a minor irritation that may produce a rash. In further aspects, a scraping or blading head can be used providing myofascial release, relieves muscle pain and tension, reduce pain and improves overall mobility, helps shear and stretch tissue, and/or increased blood flow to the treatment area. Even though a distinction is made herein between manipulation and blading, in some embodiments of the invention, the disclosed devices may deliver both manipulation and blading during operation.

In further aspects, the contact elements may have a uniform arrangement on the application area. In still further aspects, the contact elements may have a length in the range of from about 0.01 inches to about 6 inches. In yet further aspects, the contact elements have a thickness in the range of from about 0.01 inches to about 6 inches. In even further aspects, adjacent contact elements may have a spacing in the range of from about 0.01 inches to about 12 inches. In still further aspects, at least one contact element may be contained within the application area, the contact element capable of manipulating the surface of the subject, wherein manipulating comprising exerting mechanical force or pressure on tissue at the site of the subject. In some aspects, the mechanical force or motion may comprise linear motion, reciprocations, strokes, thrusts, rubbing, kneading, vibrations, oscillations, mechanical energy or motion or the like.

In further aspects, the force or motion may be a single cycle reciprocation or multiple cycles, or a combination thereof. In yet further aspects, the reciprocations per minute can be constant or variable, or a combination thereof. In still further aspects, the reciprocations are from greater than 0 to about 2, or 4, or more reciprocations or oscillations per minute. In various aspects, the force or pressure level or intensity of the device may be defined by reference to the strength and the frequency of the reciprocations. To this end, the strength is characterized as the force or pressure level that the user feels when in contact with the head attachment. The strength level of reciprocations in disclosed embodiments, according to some aspects, may be sorted into distinct levels such as low, medium, and high, and/or 0% to 100%, and/or even to allow for a selection of a level along a continuum between low and high.

The head attachment casing or body may be manufactured of a rubber or other flexible polymer material, a silicone-based material, or may be a rigid material, such as a plastic or metal. In one aspect, the materials may comprise both anodized aluminum and Shore 60A silicone. In other aspects, the materials may comprise materials such as plastics, rubbers, or metals that are known to not illicit contact dermatitis.

In various aspects, a casing of the present invention can be any shape and may be in the shape of a three-dimensional polygon. In some aspects, the casing may be solid, and in other aspects, the casing may be hollow where the casing walls may define an interior space or interior sections for containing various operating elements of the invention. Any other shape (as used herein, the term shape is used in the broad sense of three-dimensional works) may be employed, so long as the shape is large enough and structured so as to be able to contain the various working components of the invention as more fully disclosed below.

In further aspects, the head attachment may comprise a casing having an application area configured to transmit pressure and/or thermal effects to the tissue of the subject. In still further aspects, application area may comprise the portion of the casing for contacting the surface of the subject or for contacting a thermal element that in turn contacts the surface of the subject. In still further aspects, the application area may be all or a portion of the proximal side of the casing.

In some aspects, a thermal element may cooperate with the application area, such as the contact elements or tips, to apply cold or heat to the subject. The placement of the thermal element is variable so long as the effects of the thermal element can be felt on the subject so as to produce thermal vasodilation or vasoconstriction. When, utilized, the placement of the vibrational source in the device is also variable so long as the vibrational effects of the vibrational source can be felt on the subject so as to produce vibrational vasodilation or is effective in stimulating the underlying tissue. In some aspects, the walls of the casing define an interior space that, may be sized to contain at least one thermal and/or vibrational element, wiring to connect the at least one thermal and/or vibrational element. In other aspects, the casing may be solid and contains thermal elements built in that diffuse heat into the tips on the application area. In further aspects, a stainless-steel mesh can be used that acts as a power-dissipating resistor when a voltage is applied. Nichrome wire can be used for heating or other resistive elements. Peltier modules could also be used to heat or cool the tips.

In further aspects, vibration or oscillation effects can be provided by any desired vibrational element such as, for illustrative purposes, a vibratory motor provided within the casing. Once vibration is activated by providing power to the vibratory element, such as a vibratory motor, the vibration may be constant and continual, or the vibration may be intermittent, and cycle on and off at the same or a different vibration speed or frequency.

In further aspects, the device may also comprise one or more vibration elements. In still further aspects, the device may comprise a plurality of vibration elements. In yet further aspects, the size of the area of the user's body against which the vibration vibrates may depend upon the size of the vibration element of the device. For example, a larger vibration element in a vibration device may vibrate against a larger area of a user's body than a smaller vibration element in a vibration device according to this embodiment. The vibration elements of the embodiments of the invention may be of any technology or means that achieves the function of massaging and reciprocatingly manipulate the skin, fascia and muscle tissue of the subject (and in an embodiment directly against the skin of the user). In some aspects, reciprocation may be defined to be similar to a piston-like back and forth motion. In other aspects, “massage” may be understood to include more circular action or curve-like motion than piston-like linear motion of the propulsion source. Even though a distinction is made herein between reciprocated manipulation and massage, in some embodiments of the invention, the disclosed device may also deliver massage as during operation. Other embodiments of the invention may provide for the device to switch between reciprocated manipulation and massage.

According to other aspects, the invention may provide for more than one level of reciprocating linear motion for the devices. For example, a system according to an embodiment of the invention may include one or more devices with one or more of these devices having more than one level of reciprocating linear motion. Advantageously, in embodiments of the invention that include one or more devices having more than one reciprocating linear motion level, the user of the embodiment may select the level of reciprocating linear motion for the devices in the system. As further described herein, the reciprocating linear motion level selection functionality available to a user may vary from embodiment to embodiment. For example, in further aspects, a user may be able to select a level of reciprocating linear motion, but the same level is selected for all of the devices whether the system includes one or more devices. In this aspect, all the devices of that particular embodiment reciprocate at the same level. As another example, in other embodiments, a user may be able to select varying or different respective levels of reciprocating linear motion in a system having more than one tissue manipulating devices.

In various embodiments, the tissue manipulating device may include multiple means for adjustability of the position and angle of the propulsion source and/or head attachment in order to treat different anatomical regions of different people. In one aspect, device can be adjustably positioned anywhere along the central frame member of the frame assembly by translating the attached portion of the device up and down. In some embodiments, the device may be positioned anywhere along the central frame member. In other embodiments, the devices may be positioned only on a designated portion of the central frame member. In further aspects, the invention may advantageously include embodiments having different features with respect to the placement of the devices along the frame member, and various angles it extends out from the central frame member. The device can comprise an arm which can be positioned downward at any desired angle, horizontal, and/or upward at any desired angle. The head attachment and/or propulsion source can rotate about a 360-degree axis at the end of the device arm. To this end, a user may be able to choose where to position the device with respect to his or her height and body location. Wherever placed on the frame assembly, the position and/or configuration of a device optimally does not interfere with the user's ability to maintain contact with the application area of the casing.

In some embodiments, the invention may provide an app on a wireless device configured to communicate with the devices and device components. In further aspects, each of the devices may require the appropriate elements to receive, act on the communications from the wireless device, and/or respond to the app with information. As described in further detail herein, in other embodiments, the device may include a controller to receive the communications from the wireless device, to pass them on as instructions to the device, to receive information from the devices, and/or to send the information to the app on a mobile device. The controller may include elements to carry out its function. For example, the controller may include a receiver/transmitter, transceiver, and/or antenna for communicating with the wireless device. The controller may include “smart” technology such as a microprocessor, etc. to process and execute the instructions, information, and/or signal received from the control unit or wireless unit or the information received from the device. As another example, the controller may be connected respectively by one or more wires (and/or other transmitters or carriers) to the one or more devices to transmit instructions/information to the device and/or to receive information from them.

In various aspects, the controller and/or devices may require a power source depending on the configuration and construction. An exemplary power source may any type of power source such as but not limited to a connection to an alternating current source (a wall plugs), a solar or other light cell, a miniature reactor, a mechanical source such as a flywheel or springs, a disposable or rechargeable battery or the like. Other power source(s) are possible. Any suitable power source may be used.

In various aspects, the controller and/or device components may be included in or integrated within various portion of the frame assembly or casing. In further aspects, the controller and/or device components may be attached to or contained in the frame members of the frame assembly. In still further aspects, the controller and/or device components may be contained in a housing attached to a frame member or frame assembly. Other locations are possible, and may depend on the size and/or other characteristics of the controller, etc. In further aspects, the device elements and/or components can be connected to an outer surface of the device casing. In still further aspects, the device elements and/or components can be connected to an inner surface of the device casing. In yet further aspects, the device elements and/or components can be integrally connected to portions of the device casing, housing, and or frame assembly. In various aspects, the device arrangement and construction of the systems can be configured to correspond to areas of a subject's body where muscle and fascia tissue are greatest. In further aspects, the device may further comprise on opening through a wall or outer surface of a housing or frame member for providing an amplifier that is connected to the controller or a sound element contained within the casing. In still further aspects, the device may comprise an opening through the outer surface for providing a light, such as an LED light, that is connected to the controller or a timing element contained within the casing. A light (and/or sound) may be turned on when linear motion or movement is initiated and turned off when power to the device element is turned off. Alternatively, powering on the device may also power on a timing element, and optionally a light (and/or sound), so that when a desired time period has occurred, the timing element may turn off the light (and/or sound), or may turn off a light (and/or sound) and the propulsion source, or the timing element or controller may turn on sound or light after a period of operation. Alternatively, the timing element may be under a control that is separate from a control for the propulsion source. Components for switches, controller, such as a polycarbonate circuit board and the programming to accomplish the disclosed activities and others, and elements such as timing elements, sound elements and lights, are known, and can be selected or commercially acquired by those of skill in the art. Wires for connecting the elements within the devices or on the surface are contemplated by the present invention.

In some embodiments, a system of the present invention may comprise a plurality of tissue manipulating devices. The plurality of devices may comprise at least two devices, for example, two, three, four, five, or six devices, or more. In even further aspects, at least one device can be connected at a location on the frame assembly corresponding to an upper portion of the subject body. In still further aspects, at least one device can be connected at an upper portion on the frame assembly a location corresponding to a lower portion of the subject's body. In further aspects, the position of the devices can be configured or changed with respect to the frame member to permit changes to the treatment characteristics. For example, positions of the devices can include at least one: one on front or back of thighs, one on calves, triceps, or gluteus, such as, and without limitation: positions that primarily are associated with damaged skin, fascia and muscle tissue.

In further aspects, the device component characteristics and configuration, such as, for example, size and dimensions, can be configured to adjust for an individual user's weight, height, size, tissue condition, and other factors to achieve optimal load balancing and distribution. In further aspects, features of the device and components may be configured or utilized to set and/or control mechanical motion or force characteristics. For example, at least one of the following may be configured: the type of material used for the head attachments casing; the contact element shape and dimensions (e.g., height, width, thickness, surface-contacting area, etc.); the flexibility or “stretchiness” of the head attachment material; the pressure transmission characteristics of the head attachment casing and application area surface contact profile; the space between the contacting portions of the application area; and the extent of exterior surface coverage of surface-contacting application area, and the like. In other aspects, the head attachments may be able to be immersed in water to be cleaned.

In further aspects, and as described and disclosed herein, the head attachments can be removably coupled to the propulsion source to allow interchange and/or replacement. In some aspects, the tissue manipulation devices can be detachably connected to the frame member, e.g., to allow interchange and/or replacement of the propulsion source (individually or as a unit with head attachment). Such configurations allow users, purchasers, retailers, or others to select desired manipulation effects and treatment properties or levels, e.g., for customization purposes, for personal preferences, to match desired treatment use, a user's physical characteristics, a user's symptoms, or to repair or replace defective or damaged devices, etc.

In various aspects, components and parts of the disclosed devices and systems can be detachably attached. In further aspects, the components and parts can be connected by a connecting means. In still further aspects, the connecting means can comprise a joint, ball joint, fitting, insert, adhesive, brazing, soldering, welding, spot weld, screw with nut, rivet, threading, friction fit, snap-fit, twist-lock, or interlocking mechanism or a combination thereof. In yet further aspects, the connection can be achieved using a snap, friction fitting, snap ring, O-ring, pressure fitting, clip, clasp, and the like. The snap ring or O-ring can be retained within a groove to accommodate the snap ring or O-ring. In a further aspect, the system can comprise an engagement means for coupling and holding components together. In a further aspect, the engagement means can be a screwing mechanism, a click-lock mechanism, or friction mechanism, or the like. In a still further aspect, the device and system components can be integrally or mechanically attached to other components. In a yet further aspect, the disclosed components can be secured, connected, attached, or mounted using a securing means, the securing means comprising a clip, pin, clasp, fastener, fitting, insert, adhesive, brazing, soldering, welding, spot weld, screw with nut, rivet, fitting, insert, threading, friction fit, or snap-fit or a combination thereof.

In further aspects, a frame assembly of the present invention may comprise a base component to stabilize and secure the frame member during use, for example, a base (e.g., standing steel base platform) or legs for support during use. In some embodiments, the device may comprise a platform for various size individuals to stand on with optional rubber mat on top. In further aspects, the main center vertical frame member may be secured to the base or legs. The frame member may be used to attach, position and/or guide the interchangeable devices within various height settings. In still further aspects, multiple levels of stability hand rails may be attached for support on both sides which may fold up or completely collapse down. In yet further aspects, movement of the device along the frame assembly may be vertically up and/or down the frame member, for example, using a releasable locking mechanism. In even further aspects, the locking positions along the frame member may be spaced 2 inches or less apart.

In further aspects, attached device components may also releasably lock at various angles, such as, and without limitation, from 0 degrees to about 180 degrees, facing up, 90 degrees straight forward and 45 degrees facing down. In still further aspects, movement of interchangeable head attachments can comprise the ability to rotatably adjust the head attachment casing portion in degree increments up to 360-degrees. In yet further aspects, the devices may be easy to move mechanically back and forth with various distance settings anywhere on the frame member. In some aspects, the distance settings may range from 0.5″, 1″, 2″, 3″, 6″, 9″, 12″, 15″ or 18″ or more, and any distance between. In other aspects, the device may have ability to set different speed ranges for the device movement, oscillation, and/or vibration.

In further aspects, application software (“an app”) on a wireless device such as a mobile phone may be used to activate and de-activate and/or otherwise control the operation and forces emitted by the one or more devices in the system. In yet further aspects, the disclosed systems may be equipped with wireless technology to communicate with the app on the wireless device. In even further aspects, the app may provide at least ones of: a graphic image of the user's body to indicate where (and/or other characteristics of the vibrations) the device(s) are delivering vibrations to the user's body at the time of operation, possible areas of the user's body to indicate where (and/or other characteristics of the vibrations) the device(s) may be made to deliver treatment, telemetry data received from the sensors in the devices.

According to various aspects of the disclosure, the devices and systems of the present invention provides a number of advantages of current options. Without wishing to be bound by a particular theory, the combination of inventive configurations, device construction and positioning allow the disclosed devices and systems to be effective in stimulating skin, fascia and muscle tissue. In further aspects, the disclosed systems with one or more devices can selectively operable to encourage blood flow to the area, which allows unhealthy skin, fascia and muscle tissue to be internally stimulated.

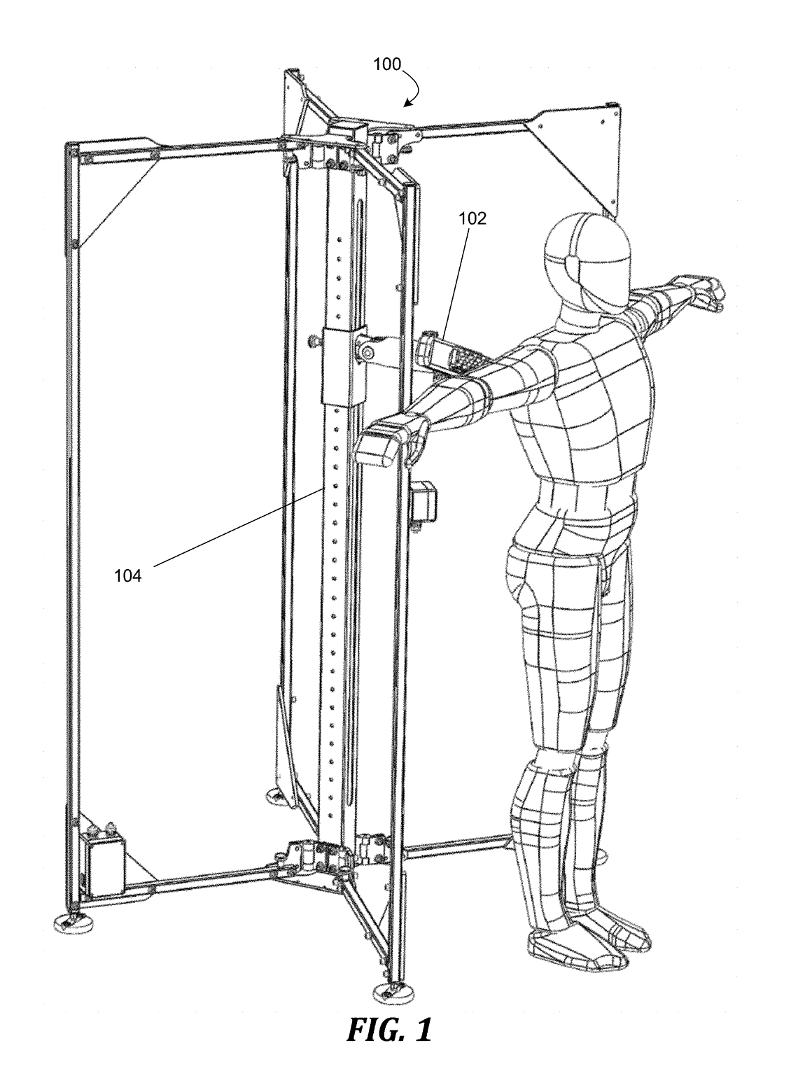

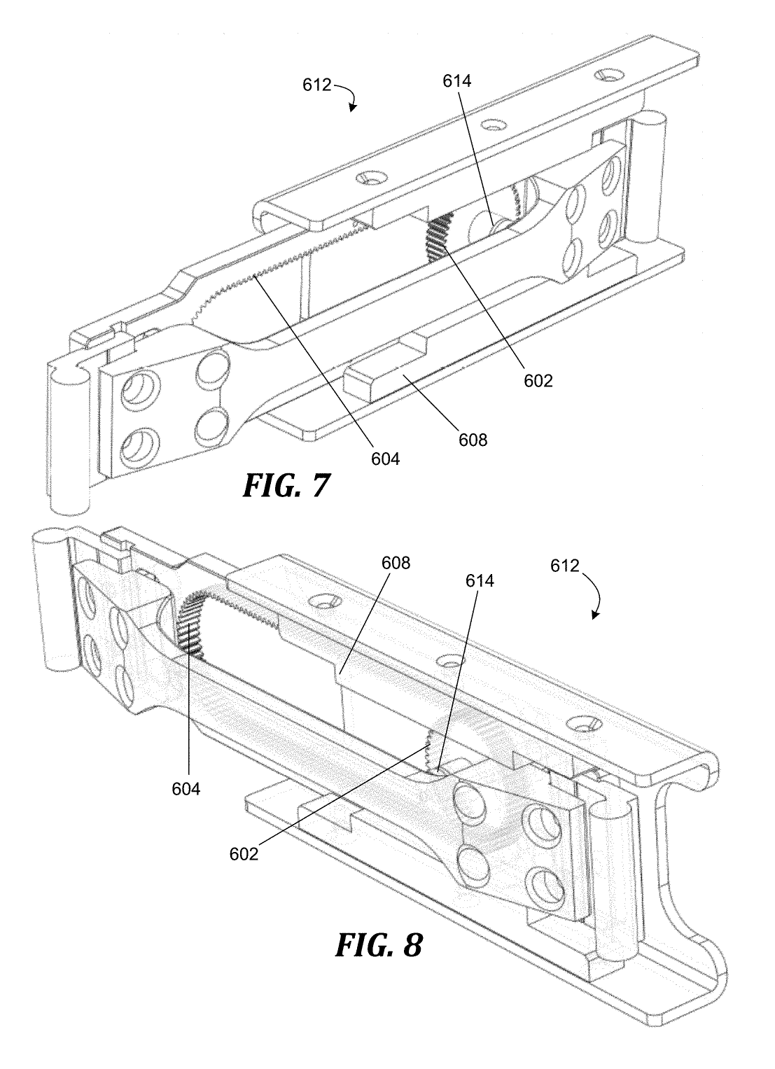

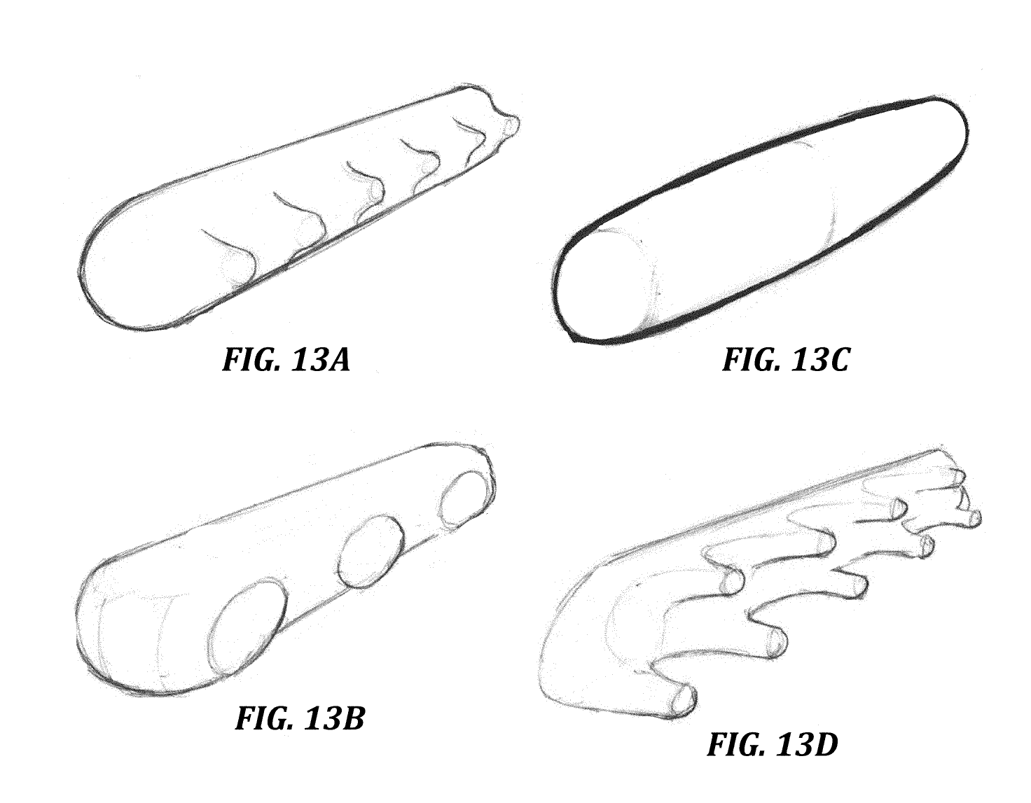

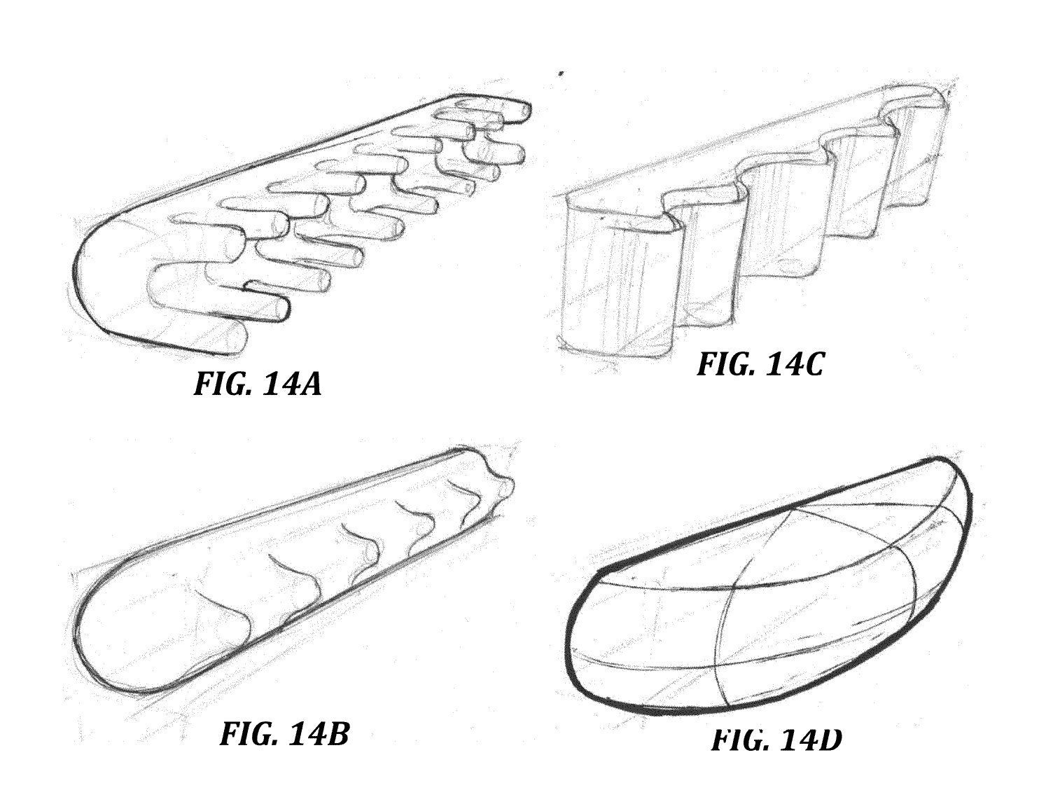

According to various aspects of the invention, the devices, methods, and systems of the present disclosure can comprise multiple configurations. FIGS. 1-18 show depictions and perspectives of exemplary embodiments of devices, methods and systems in accordance with the present invention.

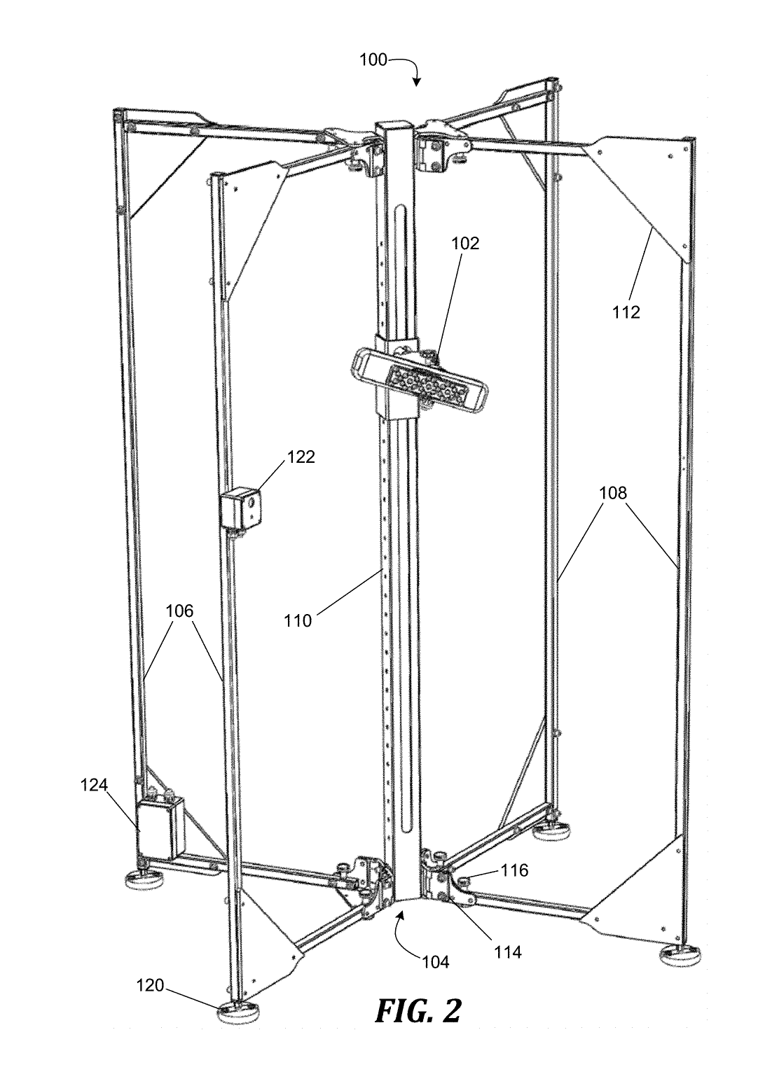

FIGS.1-2 show an exemplary embodiment of free-standing system 100 for manipulating muscle and fascia, including electromechanical tissue manipulation device 102 adjustably mounted to frame assembly 104. As shown in FIG. 2, frame assembly 104 comprises left side frames 106 having first and second left side frames and right-side frames 108 having first and second right side frames, both side frame pairs connected to vertical frame member 110. In further aspects, the side frame can comprise a plurality of frame members connected together to form the side frame, for example, first frame members and second frame members, each having first and second opposed ends. First frame members may be substantially equal in height to central frame member, each end connected to first ends of upper and lower second frame members. The frame assembly can comprise at least one cross support plate at each end connecting the first frame member with each of the second frame members, such as the triangular plate 112 shown in FIG. 2. In further aspects, frame assembly 104 is shown in the expanded state, first and second left side frames forming a substantially 90-degree angle, as do first and second right side frames. Each side frame is connected to the central frame member using hinged connector 114, and secured at an expanded state position or collapsed state position using pin locking mechanism 116.

Still further, each side frame may comprise a foot for stabilizing the frame assembly. FIG. 2 shows an exemplary foot 120 including adjustable foot mounting, and foot gripping area. The adjustable foot mounting is configured to allow adjustment of the height of the foot. In further aspects, the foot and/or foot gripping area can be comprised of any suitable material, such as, for example, plastic, rubber, high density cushioning foam, foam and fabric, or other material adapted for ground impact. In some aspects, the foot and/or foot gripping area may also comprise a tread pattern to provide traction. In other aspects, the foot may contain a flat bottom. Controller 122 and power source 124 may be contained in housings attached to the frame assembly.

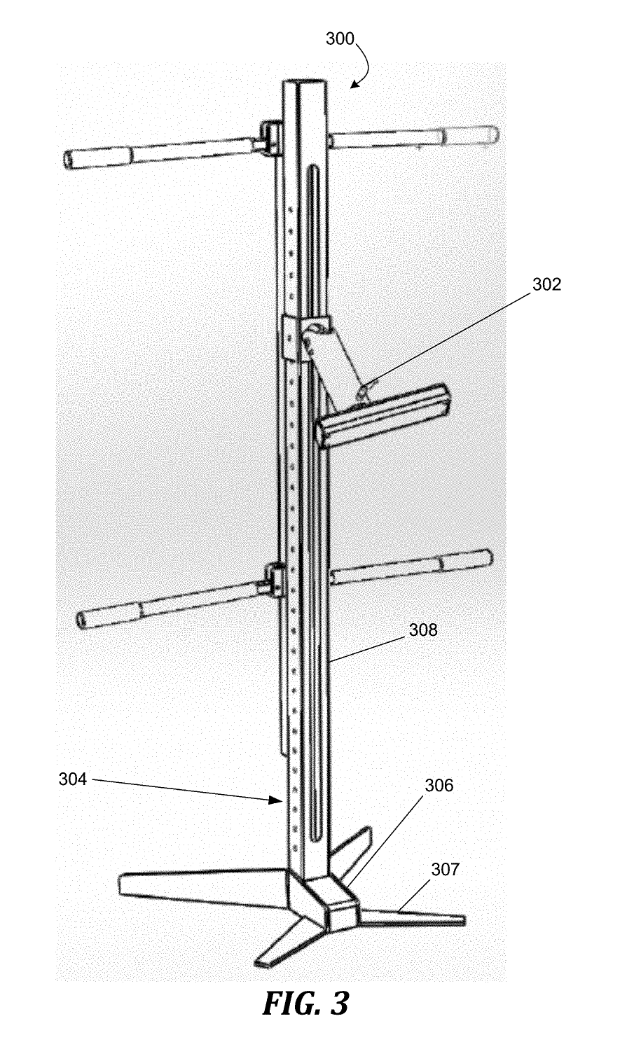

FIG. 3 show another exemplary embodiment of free-standing system 300 for massaging muscle and fascia, including electromechanical tissue manipulation device 302 adjustably mounted to frame assembly 304. Frame assembly 304 comprises base component 306 with legs 307 secure with center vertical frame member 308. Center vertical frame member 308 may be used to attach, position and/or guide device 302 within various height settings.

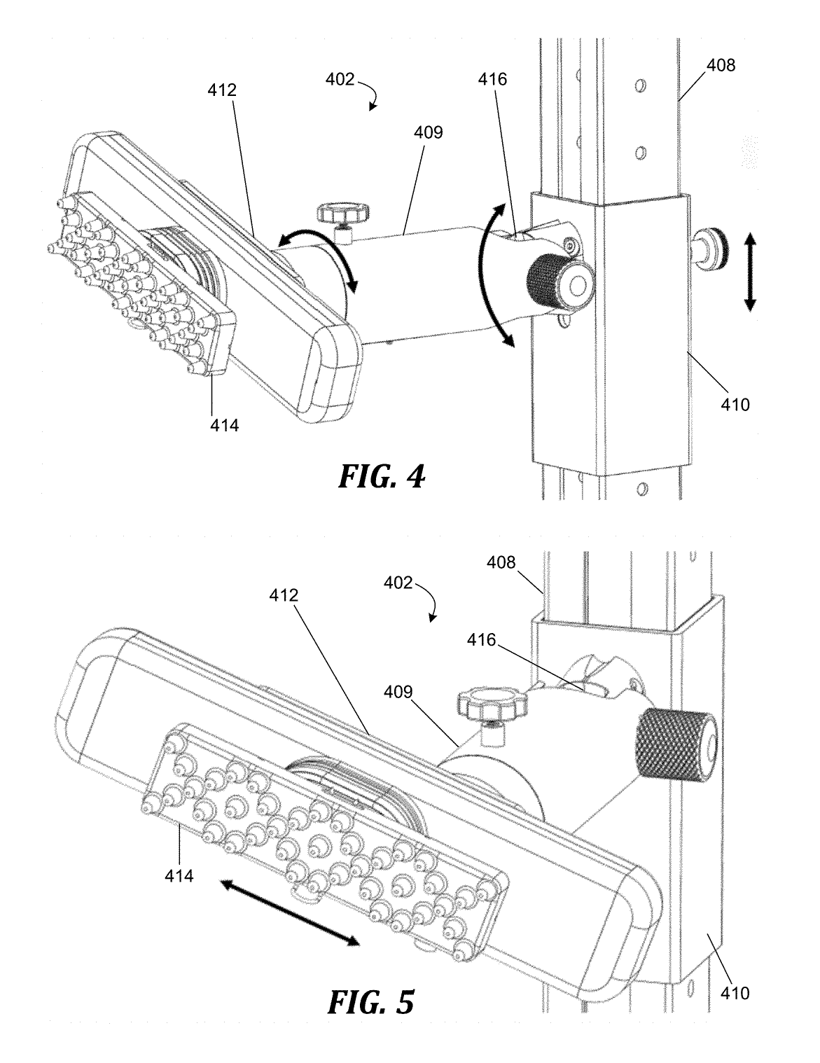

FIGS. 4-5 show another exemplary device 402 for manipulating tissue and fascia, adjustably connected to frame member 408 via device arm 409 and frame member mount 410. Device 402 includes reciprocating mechanism 412 securing interchangeable manipulating head 414 that interfaces with the user's anatomy and tissue. Reciprocating mechanism 412 manipulates using a reciprocating linear motion with four inches of travel. The speed may be user-adjustable and can range from zero to two cycles per second.

Reciprocating mechanism 412 housing can rotate about the axis of the motor contained with the device arm 409. The device arm 409 can rotate up or down about a pivot point 416 near the frame member mount 410, which can translate up and down frame member 408 that it is mounted on.

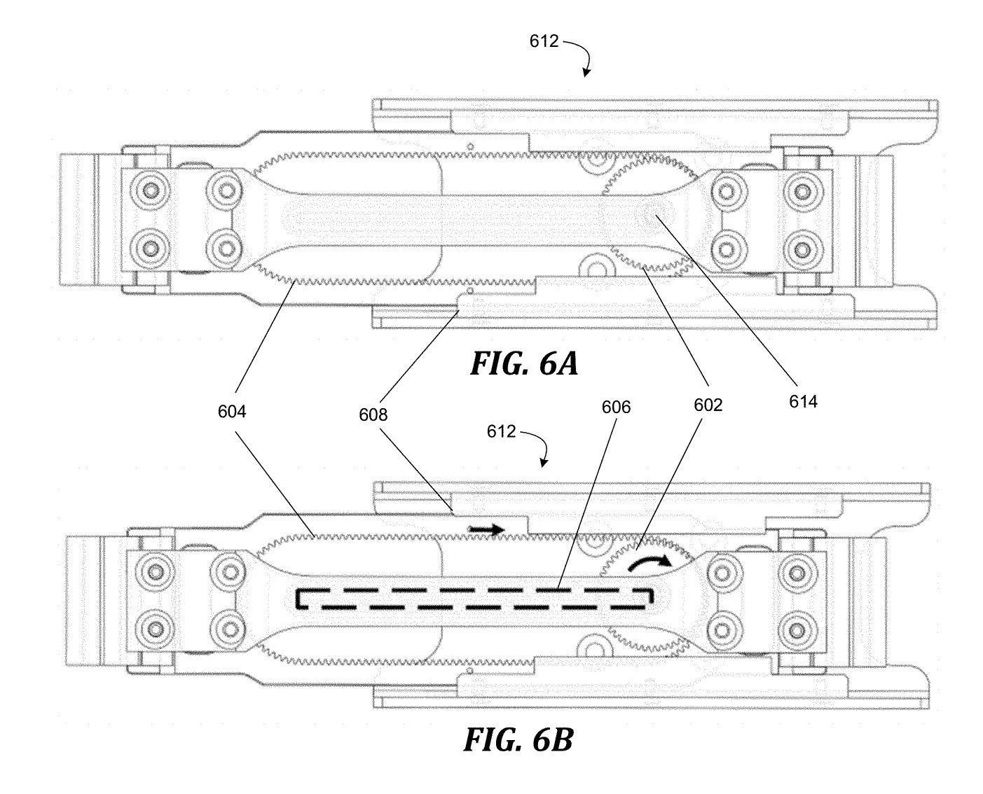

FIGS. 6A-8 show various depictions of components and configurations showing an exemplary reciprocating mechanism 612 for producing effective reciprocating linear force in accordance with the present invention. As shown, internal components of reciprocating mechanism 612, including gear 602, involute slot gear rack 604, and path guide 606, and linear guides 608. These components are contained in a housing or enclosure behind the manipulating head connection. As shown in FIG. 6B, reciprocating mechanism 612 allows for conversion of continuous-direction rotary motion to reciprocating linear motion. DC motor 614 drives the pinion gear 602. As the pinion gear 602 is engaged with the involute slot gear rack 604, a pin attached to the pinion 602 must follow the guide track, the assembly will follow the track and move side-to-side. Vertical linear guides 608 on the side maintain orientation of the involute slot gear rack 604.

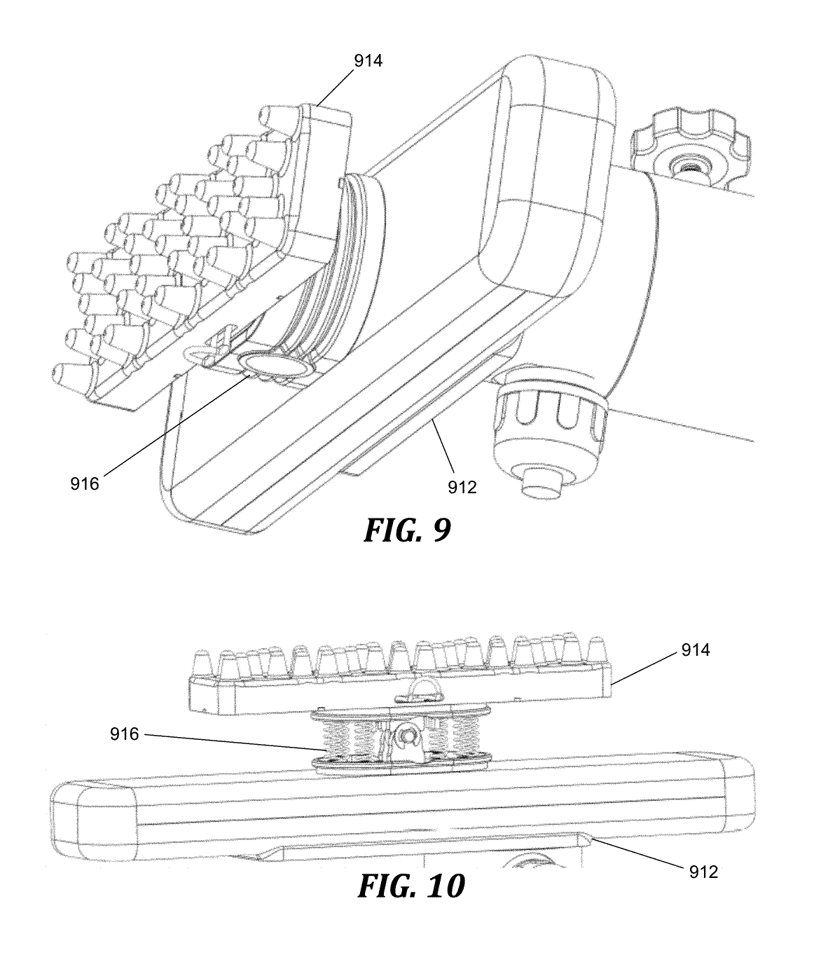

FIGS. 9-10 show interchangeable manipulating head 914 detachably connected to a propulsion source 912 in accordance with another exemplary embodiment of the present invention. Manipulating head 914 may be connected to propulsion source 912 via spring suspension system 916. As shown in FIG. 10, the removed bellows reveal the mechanism of a spring suspension system 916 which includes a one degree of freedom hinge along with multiple springs. This mechanism may allow for the device to comply to various types of anatomical geometry and provides comfortable compliance for the manipulating head 914.

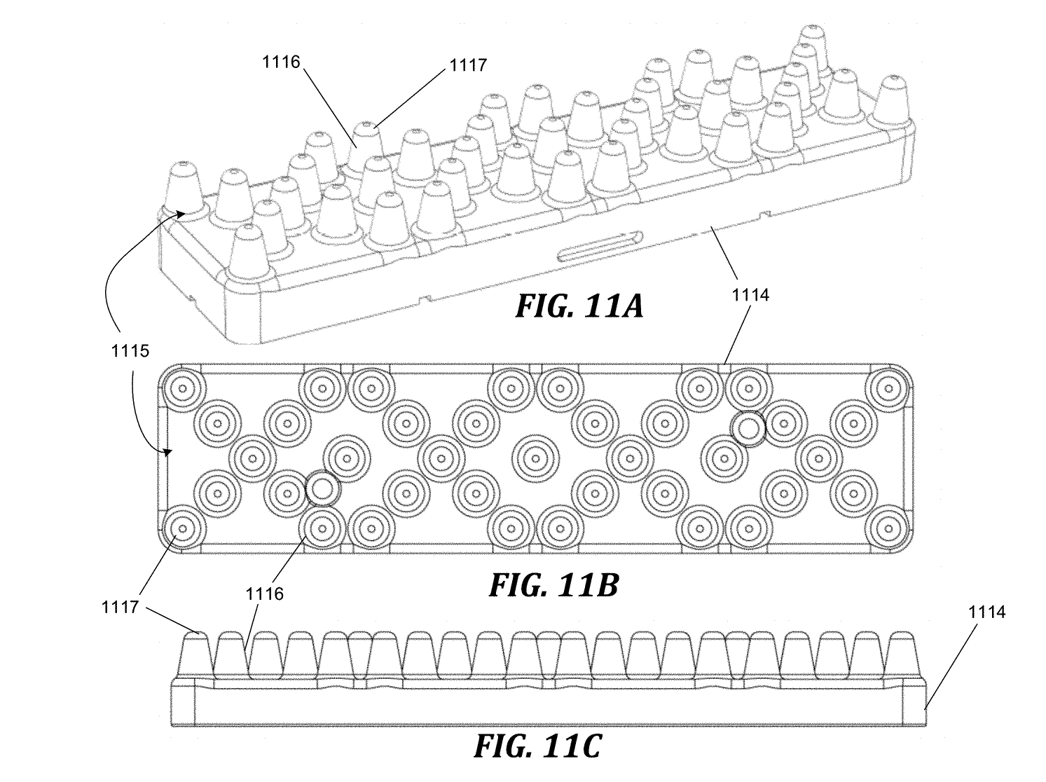

FIGS. 11A-14D show various manipulating heads with varying contact surface profiles for use in connection with various types of skin, fascia and muscle tissues in accordance with other exemplary embodiments of the present invention. FIGS. 11A-11C show various view of an exemplary manipulating head 1114 with contact surface profile 1115 having a plurality of contact members 1116 for manipulating and stimulating tissue. The contact members 1116 may further comprise a thermal application area 1117 for administering thermal effects such as heat (or cool) simultaneously with tissue manipulation. As will be detailed below, a thermal source contained within the device may cooperate with contact members 1116 to diffuse heat through thermal application area 1117 to the tissue.

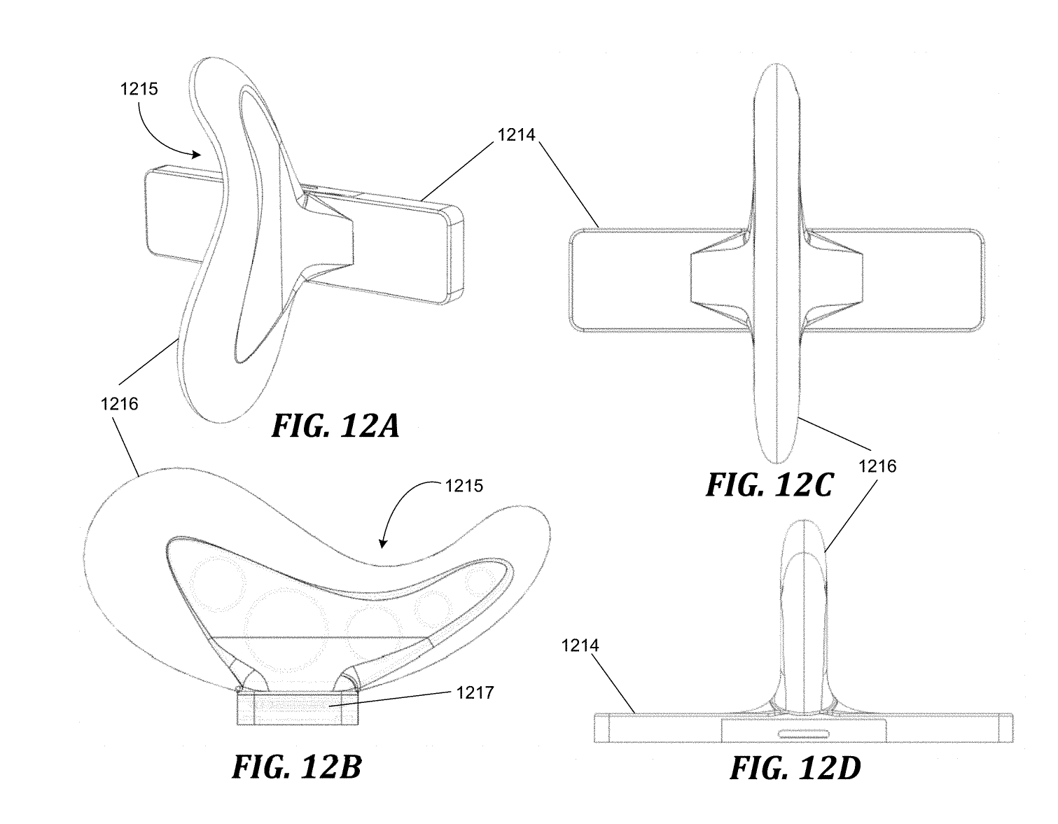

FIGS. 12A-12D show various view of an exemplary blading head 1214 with blade element 1216 for use in connection with “blading” tissue techniques as described herein. Blade element 1216 has a contact surface profile 1215 including a combination of smooth, organic concave and convex contours. Blading head 1214 may be made of a firm rubber that can allow for comfortable deep massage of muscle and fascia. As shown in FIG. 12B, rubber can be overmolded around a rigid metal (e.g., aluminum) core support 1217 which can provide rigidity for tissue penetration while the softness of the rubber can provide comfortable contact.

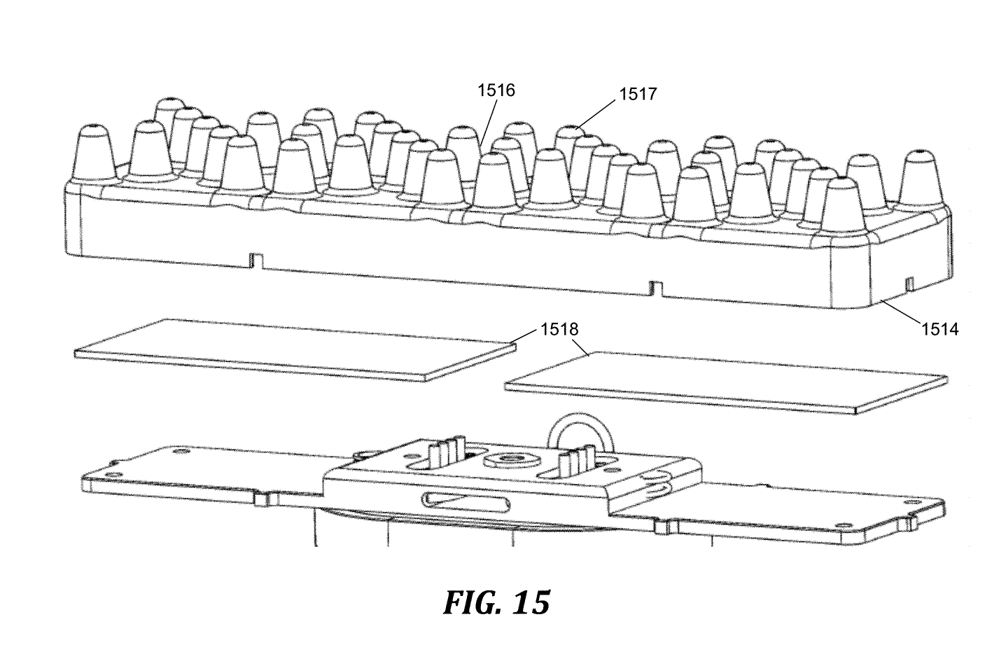

As described herein, the disclosed devices and systems may comprise a thermal source capable of producing thermal effects, such as, for example, a temperature modulation system. FIG. 15 shows an exploded view of manipulating head 1514 configured to connect to a thermal source and cooperate with manipulating head contact members 1516 to diffuse heat through thermal application area 1517 to provide a comfortably warm interface with the user's skin or as desired. As shown, the thermal source can be a regulated heating system comprising heating pads 1518 whose inherent resistance dissipates heat through manipulating head 1514 when in contact with heating pads 1518. A temperature sensor may provide feedback to the controller to regulate the temperature.

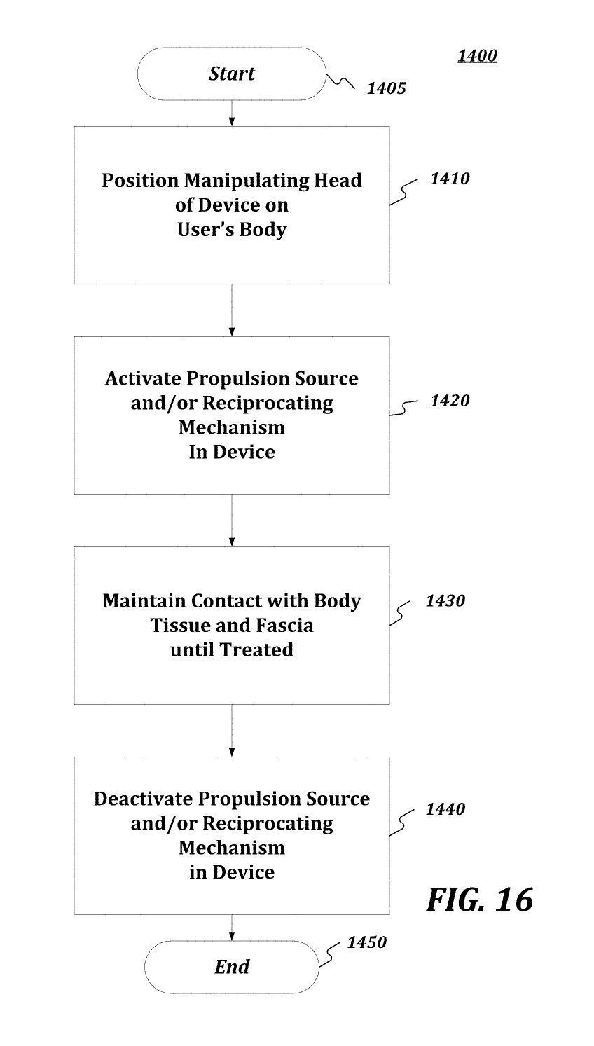

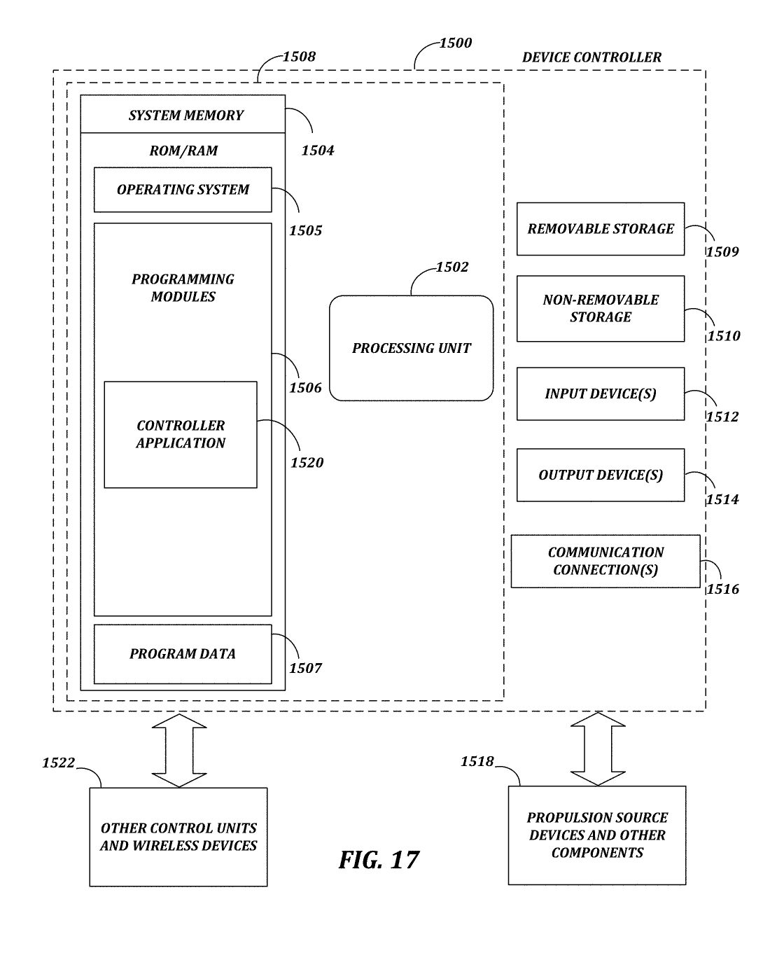

Also disclosed herein are methods of using the disclosed devices and systems. For example, in another exemplary aspect, the present disclosure provides a method for treating damaged skin, fascia and muscle tissue in a subject using a disclosed device or system. In further aspects, FIG. 16 is a flow chart setting forth the general stages involved in a method 1400 consistent with an embodiment of the disclosure for operating the disclosed devices and systems. Method 1400 may be implemented using system 100 including tissue manipulation device 102 and frame assembly 104 described in FIGS. 1-2, and, at least in part, a controller 1500 (e.g., microcontroller or on-board computing device) as described in more detail below with respect to FIG. 17. Controller 1500 may comprise a controller for operating the devices and device components as well as well as performing other operational tasks, including, but not limited to, reciprocating motion control and parameters, and communication. As such, controller 1500 may be in operative configuration and communication with, for example, but not be limited to, motors, thermal elements, activating switch, communication module, power source, power regulator, various telemetry sensors, transceivers and antennas. As will be detailed with reference to FIG. 17, controller 1500 may comprise a remote communication module to enable remotely operation as described herein. In other embodiments, controller 1500 may be completely self-operating upon configuration.

Furthermore, although stages are disclosed with reference to controller 1500, it should be understood that a plurality of other components may enable the operation of method 1400, including, but not limited to, other computing components, mechanical components (e.g., manipulating head attachments), environment properties (e.g., temperature), user conditions, and the like. Further still, although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein.

Method 1400 may begin at starting block 1405 and proceed to stage 1410, where the device may be secured against or positioned on a surface of a user's body, as shown in FIG. 1. From stage 1410, where the device is positioned on the user's skin, method 1400 may proceed to stage 1420 where the device and/or components may be activated. The activation of device components, though disclosed in a particular order for illustrative purposes, may occur in other arrangements. Upon activation, one or more of reciprocating elements may begin to transmit reciprocating mechanical forces to the underlying tissue and/or fascia at the site where the device is contact with. In some embodiments where present, one or more thermal elements may be activated to provide heat and/or cool to the same tissue.

In various aspects, an advantage of the invention can be that it allows a user to remotely activate the devices and effectively self-administer treatment without extreme and to areas of the body that normally cannot be accessed during self-administered treatment. In further aspects, a user does not have to physically pick up or hold the device to: activate its operation, administer treatment, shut off operation, and/or in embodiments that allow for adjustment, to adjust the level. In still further aspects, a user may activate or de-activate (and/or otherwise control operation of) the disclosed devices by using a control unit, such as a wireless device or mobile device that is in operative communication with the disclosed device. The wireless device may be a device that may be used for additional purposes other than use with the invention such as a mobile phone, tablet computer, notebook computer, desktop computer, etc. In an embodiment, the invention may provide a specialized wireless device for use with the invention. The specialized wireless device may include other uses if its use is not limited to this particular embodiment of the invention. As provided in more detail herein, the control unit used to control the devices may include an application or application software (an “app”) specifically created for such usage. Advantageously, the user may download and/or otherwise obtain the app from sources that supply apps such as independent developers and app stores. The app as used with embodiments of the invention communicates wirelessly, such as by using Bluetooth or the like technology.

Controller 1500 (e.g., microcontroller or on-board computing-device) may automatically activate device elements instantly or after a set amount of time has passed since the launch. In other embodiments, activation may occur upon certain reading from on-board sensors (e.g., including, but not limited to, sensors deployed in the casing, housing and/or frame assembly). For example, activation of one or more reciprocating and/or thermal elements may be dependent on certain environmental factors and/or user conditions such as, for example, temperature, pressure, acceleration, and the like. Controller 1500 may be configured to trigger activation of device components upon the satisfaction of certain pre-set conditions. Such conditions may be defined prior to activation.

From stage 1420, where the device components are activated, method 1400 may proceed to stage 1430, where the device will deliver the reciprocating mechanical forces and/or thermal effects to the tissue site for a time sufficient to restore fascia and/or muscle tissue health. From stage 1430, where the device is used to perform treatment to the tissue, method 1400 may proceed to stage 1440, where the device components are turned off. After stage 1440, method 1400 may end at stage 1450. During all stages, the devices may be in operable communication with the user via wired or wireless communication component. The user may receive various readings from the various device components. In some embodiments, the user may control the operation of the devices during use. For example, the user may be able to control the device components, including, but not limited to, reciprocating elements, thermal elements, activating switch, communication module, power source, power regulator, various telemetry sensors, transceivers and antennas. In other embodiments, controller 1500 may be pre-configured with operational control instructions and/or data. In further aspects, embodiments of the disclosed devices may be used for treating pain and unpleasant sensations in the tissue.

In various aspects, the disclosed devices and systems may comprise, but not be limited to, an integrated controller and/or on-board computing module. The computing module may be in operative configuration and communication with, for example, but not be limited to, reciprocating elements, thermal elements, activating switch, communication module, power source, power regulator, various telemetry sensors, transceivers and antennas. Further, the computing module may be in operative communication with another computing device consistent with the description herein, and may comprise, but not be limited to, a wireless device, smart phone, desktop computer, laptop, a tablet, or mobile telecommunications device. Such remote devices may be used to control and/or configure integrated computing module (e.g., activation conditions, reciprocating force operating parameters and settings, and the like). Moreover, the devices may be in operative communication with a centralized server, such as, for example, a cloud computing service. Although operation has been described to be performed, in part, by a controller 1500, it should be understood that, in some embodiments, different operations may be performed by different networked elements in operative communication with controller 1500.

Embodiments of the present disclosure may comprise a system having a memory storage and a processing unit. The processing unit may be coupled to the memory storage, wherein the processing unit is configured to perform the stages of method 1400. FIG. 17 is a block diagram of a system including controller 1500. Consistent with an embodiment of the disclosure, the aforementioned memory storage and processing unit may be implemented in a computing device, such as controller 1500. Any suitable combination of hardware, software, or firmware may be used to implement the memory storage and processing unit. For example, the memory storage and processing unit may be implemented with controller 1500 or any of devices and components 1518, or any other control unit and wireless devices 1522, in combination with controller 1500. Other devices and components 1518 may comprise, for example, but not be limited to, control mechanisms, vibrational elements, thermal elements, activating switch, communication module, power source, power regulator, various telemetry sensors, transceivers and antennas. The aforementioned system, device, and processors are examples and other systems, devices, and processors may comprise the aforementioned memory storage and processing unit, consistent with embodiments of the disclosure.

With reference to FIG. 17, a system consistent with an embodiment of the disclosure may include a computing device, such as controller 1500. In a basic configuration, controller 1500 may include at least one processing unit 1502 and a system memory 1504. Depending on the configuration and type of computing device, system memory 1504 may comprise, but is not limited to, volatile (e.g. random-access memory (RAM)), non-volatile (e.g. read-only memory (ROM)), flash memory, or any combination. System memory 1504 may include operating system 1505, one or more programming modules 1506, and may include a program data 1507. Operating system 1505, for example, may be suitable for controlling controller 1500's operation. In one embodiment, programming modules 1506 may include controller application (“app”) 1520. Furthermore, embodiments of the disclosure may be practiced in conjunction with a graphics library, other operating systems, or any other application program and is not limited to any particular application or system. This basic configuration is illustrated in FIG. 17 by those components within a dashed line 1508.

Advantageously, the app may provide a user with information as well as be the user's interface to operating the embodiment of the invention. The app may include one or more graphic user interfaces (GUIs). Among the GUIs of the app may be a GUI allowing the user to pick which, if there is more than one, reciprocating and/or thermal elements to activate, and to select (if available) one or more operating parameters or characteristics (such as strength or frequency) of the reciprocating mechanical forces of the head attachment(s). The user may be able to adjust such selections without having to deactivate the embodiment from a GUI of the app. The user may also use the app to turn on and turn off the device components. Another advantage of the app is that the app may present the user with a GUI that depicts the user's body (or a generic body) and shows where the forces are being applied. The GUI may include additional or other information relating to the forces being applied such as the strength or frequency. The additional or other information may be color coded and/or otherwise presented so as to be readily understood by the user by looking at the GUI of the app. The app may also present the user with information received from the device components, such as environmental and telemetry data.

Controller 1500 may have additional features or functionality. For example, controller 1500 may also include additional data storage devices (removable and/or non-removable) such as, for example, magnetic disks, optical disks, or tape. Such additional storage is illustrated in FIG. 17 by a removable storage 1509 and a non-removable storage 1510. Computer storage media may include volatile and nonvolatile, removable and non-removable media implemented in any method or technology for storage of information, such as computer readable instructions, data structures, program modules, or other data. System memory 1504, removable storage 1509, and non-removable storage 1510 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by controller 1500. Any such computer storage media may be part of device 1500. Controller 1500 may also be operative with input device(s) 1512 such as a keyboard, a mouse, a pen, a sound input device, a touch input device, etc. Input device(s) 1512 may be used to, for example, manually access and program controller 1500. Output device(s) 1514 such as a display, speakers, a printer, etc. may also be included. The aforementioned devices are examples and others may be used.

Controller 1500 may also contain a communication connection 1516 that may allow device 1500 to communicate with other control units and wireless devices 1522 as well as devices and other components 1518 (e.g., transceivers, sensors, thermal elements), such as over an encrypted network in a distributed computing environment. Communication connection 1516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, Bluetooth™, radio frequency (RF), infrared, and other wireless media. The term computer readable media as used herein may include both storage media and communication media.

As stated above, a number of program modules and data files may be stored in system memory 1504, including operating system 1505. While executing on processing unit 1502, programming modules 1506 (e.g., controller application 1520) may perform processes including, for example, one or more of stages or portions of stages of method 1400 as described above. App 1520 may be configured to operate device components 1518 and receive instructions from, for example, communications connections module 1516. The aforementioned process is an example, and processing unit 1502 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

The present invention includes at least the following aspects: Aspect 1: A device for stimulating or manipulating tissue at a site in a subject, the device comprising: a) a propulsion source capable of producing motion; b) a manipulating head attached to the propulsion source and comprising a casing with an application area, the application area having one or more contact portions configured to contact at least one site of a subject; and c) the propulsion source and the manipulating head being cooperatively structured and disposed to cause movement of the application area in response to the produced motion; and wherein the propulsion source and application area are operatively positioned relative to one another such that the propulsion source and contact portions cooperate to manipulate tissue at the site of the subject.

Aspect 2: The device of any preceding aspect, wherein the propulsion source is adjustably mounted to a frame assembly.

Aspect 3: The device of aspect 2, wherein the frame assembly comprises a plurality of side frames connected with a central frame member.

Aspect 4: The device of aspect 2, wherein frame assembly comprising a frame member having first and second opposed sides and a base connected to at least one end of the frame member; wherein the frame assembly is configured to detachably connect or attach with the propulsion source.

Aspect 5: The device of any preceding aspect, wherein the frame assembly is configured to allow an attached device to travel along the central frame member.

Aspect 6: The device of any preceding aspect, wherein the propulsion source comprises a reciprocating mechanism capable of producing a reciprocating linear motion.

Aspect 7: The device of any preceding aspect, wherein the casing of the manipulating head is manufactured from a rigid material comprising at least one of: plastic, metal and wood.

Aspect 8: The device of any preceding aspect, wherein the casing of the manipulating head is manufactured from a pliant material comprising at least one of: natural or synthetic woven or non-woven fabrics, rubbers, flexible polymer materials, and flexible silicone-based materials.

Aspect 9: The device of any preceding aspect, wherein the contact portions of the application area comprise integrated raised portions of the application area.

Aspect 10: The device of any preceding aspect, wherein the integrated raised portions have a uniform shape and/or size.

Aspect 11: The device of any preceding aspect, wherein the integrated raised portions have varying shapes.

Aspect 12: The device of any preceding aspect, wherein the integrated raised portions have a semi-circular or concave shape.

Aspect 13: The device of any preceding aspect, wherein the integrated raised portions have a sinusoidal shape.

Aspect 14: The device of any preceding aspect, wherein the integrated raised portions have a bladed contact surface profile.

Aspect 15: The device of any preceding aspect, wherein proximal ends of the integrated raised portions are rounded.

Aspect 16: The device of any preceding aspect, wherein the contact portions further comprise one or more pressure members extending out therefrom.

Aspect 17: The device of any preceding aspect, wherein the contact portions comprise a plurality of contact members attached or disposed on the application surface.

Aspect 18: The device of any preceding aspect, wherein the contact members have a uniform arrangement on the application area.

Aspect 19: The device of any preceding aspect, wherein the contact members have a length in the range of from about 0.01 inches to about 6 inches.

Aspect 20: The device of any preceding aspect, wherein the contact members have a thickness in the range of from about 0.01 inches to about 6 inches.

Aspect 21: The device of any preceding aspect, wherein adjacent contact members have a spacing in the range of from about 0.01 inches to about 12 inches.

Aspect 22: The device of any preceding aspect, further comprising at least one pressure element contained within the casing, the pressure element capable of stimulating or manipulating the surface of the subject.

Aspect 23: The device of any preceding aspect, further comprising a controller communicatively connected to the device and configured to control operation of one or more device components based on instructions regarding operation of the device.

Aspect 24: The device of any preceding aspect, further comprising a control unit communicatively connected to the controller and configured to transmit instructions regarding the operation of the device to the controller.

Aspect 25: The device of any preceding aspect, wherein the control unit comprises a switch in operative communication with the device for selectively activating one or more of the device components.

Aspect 26: The device of any preceding aspect, wherein the control unit comprises a mobile device having application software configured to transmit instructions regarding the operation of the device to the controller.

Aspect 27: The device of any preceding aspect, further comprising at least one vibrational element capable of delivering vibrations to the subject and/or at least one thermal element capable of delivering cold or heat to the subject.

Aspect 28: The device of any preceding aspect, wherein the device comprises a plurality of vibrational elements and/or a plurality of thermal elements.

Aspect 29: The device of any preceding aspect, wherein the thermal element and/or vibration element are contained within a casing of the head attachment.

Aspect 30: The device of any preceding aspect, wherein manipulating comprises exerting a pressure or force on tissue at the site of the subject.

Aspect 31: The device of any preceding aspect, wherein the motion comprises reciprocating linear motion, vibrations, or oscillations, mechanical energy or the like.

Aspect 32: The device of any preceding aspect, wherein the reciprocating motion is a single reciprocation cycle or multiple reciprocation cycles, or a combination thereof.

Aspect 33: The device of any preceding aspect, wherein the reciprocations per minute is constant or variable, or a combination thereof.

Aspect 34: The device of any preceding aspect, wherein the reciprocations are from greater than 0 to about 4 reciprocations per second.

Aspect 35: The device of any preceding aspect, wherein the reciprocations per second cycle between from about 1 to about 8 reciprocations per second.

Aspect 36: The device of any preceding aspect, wherein the reciprocating linear motion has a travel distance of greater than 0 to about 8 inches.

Aspect 37: The device of any preceding aspect, wherein the reciprocating linear motion has a travel distance of greater than 0 to about 4 inches.

Aspect 38: The device of any preceding aspect, wherein the reciprocating linear motion has a travel distance of greater than 0 to about 2 inches.

Aspect 39: The device of any preceding aspect, further comprising a plurality of manipulating head attachments.

Aspect 40: The device of any preceding aspect, wherein the plurality of manipulating head attachments reciprocates at different reciprocations per second.

Aspect 41: The device of any preceding aspect, wherein the frame assembly comprises at least one handle member pivotably connected to a portion of the vertical frame member.

Aspect 42: The device of any preceding aspect, wherein the frame assembly comprises a plurality of handle members pivotably or foldably connected to a portion of the vertical frame member.

Aspect 43: The device of any preceding aspect, wherein the handle member is pivotably or foldably connected to a portion of the frame member at a perpendicular angle when in a use position and are configured to fold at the connection point to a storage position where the handle members are substantially parallel to the vertical frame member.

Aspect 44: The device of any preceding aspect, wherein the casing of the manipulating head is pivotably connected to the propulsion source using a spring-assisted connecting mechanism.

Aspect 45: The device of any preceding aspect, wherein the casing of the manipulating head is pivotably connected to the propulsion source using a joint.

Aspect 46: The device of any preceding aspect, wherein a housing or enclosure containing the propulsion source is pivotably or rotatably connected to a device arm using a connecting mechanism.

Aspect 47: The device of any preceding aspect, wherein a housing or enclosure containing the propulsion source is pivotably or rotatably connected to a device arm using a joint.

Aspect 48: The device of any preceding aspect, wherein the joint comprises a pivot ball with an arm extending therefrom, a socket for receiving said ball, and a retaining means for securing the pivot ball in the socket.

Aspect 49: The device of any preceding aspect, wherein the casing of the manipulating head is configured to rotate or pivot about an axis to a component to which it is connected.

Aspect 50: The device of any preceding aspect, wherein a housing or enclosure containing the propulsion source is configured to rotate or pivot about an axis to a component or portion of the device to which it is connected.

Aspect 51: The device of any preceding aspect, wherein the propulsion source is configured to move the casing in a predefined manner while the application area of the casing is in contact with the subject to thereby manipulate the tissue.

Aspect 52: The device of any previous aspect, wherein the propulsion source comprises at least one of: a motor, screw, worm screw, barrel screw, wheel and axle, cam, rod, gear, track, belt, chain, rack and pinion, or the like.

Aspect 53: The device of any previous aspect, wherein the connecting mechanism comprises a pivot point, hinge, joint, ball joint, fitting, insert, adhesive, brazing, soldering, welding, spot weld, screw with nut, rivet, threading, friction fit, snap-fit, twist-lock, or interlocking mechanism or a combination thereof.

Aspect 54: The device of any previous aspect, wherein the device can comprise an engagement means or securing means for coupling and holding device components together.

Aspect 55: The device of any previous aspect, wherein the manipulating head comprises an engagement mechanism for releasably coupling to the propulsion source.

Aspect 56: The device of any previous aspect, wherein the engagement means can be a screwing mechanism, a click-lock mechanism, or friction mechanism, or the like.

Aspect 57: The device of any previous aspect, wherein the engagement means can be a clasp, clip, pin, fastener, snap, closure, or the like.

Aspect 58: A system for stimulating or manipulating tissue at a site in a subject, the system comprising: a) a device of any preceding aspect; b) a frame assembly comprising a plurality of side frames hingedly connected to a central frame member having first and second opposed sides and a base connected to at least one end of the frame member; wherein the frame assembly is configured to detachably connect or attach with the casing; c) a controller communicatively connected to the device and configured to control operation of the one or more device components based on instructions regarding operation of the device; and d) a control unit communicatively connected to the controller and configured to transmit instructions regarding the operation of the device to the controller; wherein the propulsion source is configured to move the casing in a predefined manner while the application area of the casing is in contact with the subject to thereby manipulate the tissue.

Aspect 59: A system for stimulating or manipulating tissue at a site in a subject, the system comprising: a) a device comprising: i) a manipulating head attachment having a casing comprising an application area, the application area including one or more contact portions configured to contact at least one site of the subject; ii) a propulsion source capable of exerting a reciprocating mechanical force; and iii) a thermal source or element capable of producing heat and cold; wherein the propulsion source and contact portions are operatively positioned relative to one another such that the propulsion source and contact portions cooperate to manipulate tissue at the site of the subject; b) a frame assembly comprising a plurality of side frames hingedly connected to a central frame member having first and second opposed sides and a base connected to at least one end of the frame member; wherein the frame assembly is configured to detachably connect or attach with the casing; c) a controller communicatively connected to the device and configured to control operation of the one or more device components based on instructions regarding operation of the device; and d) a control unit communicatively connected to the controller and configured to transmit instructions regarding the operation of the device to the controller; wherein the propulsion source is configured to move the casing in a predefined manner while the application area of the casing is in contact with the subject to thereby manipulate the tissue

Aspect 60: The device of any preceding aspect, wherein the contact portions comprise a surface contact profile.

Aspect 61: The device of any preceding aspect, further comprising at least one thermal element or source capable of producing thermal effects selected from at least one of heat or cold.

Aspect 62: The device of any preceding aspect, wherein the thermal element is contained with the head attachment casing or housing, or a combination thereof.

Aspect 63: The device of any preceding aspect, wherein the application area is configured to allow transfer of thermal effects from the thermal element to a surface of the site of the subject.

Aspect 64: The device of any preceding aspect, wherein the wherein substantially all of a surface of the thermal element is in thermal communication with the application area.

Aspect 65: The device of any preceding aspect, wherein at least a portion of the casing is comprised of a thermally conductive material.

Aspect 66: The device of any preceding aspect, wherein the propulsion source is in mechanical communication with the thermal element such that substantially all the mechanical force from the propulsion source is transferred through the thermal element.

Aspect 67: The device of any preceding aspect, wherein at least a portion of the application area corresponds to at least a portion of an outer surface of the thermal element.

Aspect 68: The device of any preceding aspect, wherein the thermal element is in thermal communication with at least a portion of the application area.

Aspect 69: The device of any preceding aspect, wherein the propulsion source and thermal element are operatively positioned relative to one another such that the propulsion source and thermal element cooperate to provide fascia stimulation and thermal effects to the site of the subject.

Aspect 70: The device of any preceding aspect, wherein the device is configured to provide thermal effects simultaneously with fascia stimulation; and wherein the application area is configured to allow transfer of mechanical force from the propulsion source to a surface of the site of subject.

Aspect 71: The device of any preceding aspect, wherein the device is configured to provide a thermal effect simultaneously with fascia stimulation; and wherein the application area is configured to allow transfer of mechanical force or pressure and thermal effects to a surface of the site of subject.

Aspect 72: The device of any preceding aspect, wherein the device is configured to deliver at least one thermal effect simultaneously with reciprocating mechanical manipulation that is effective to treat a site in a subject.

Aspect 73: The device of any preceding aspect, wherein the combination of reciprocating mechanical manipulation and thermal effects transmitted produces fascia analgesia and thermal analgesia treatment effective to reduce pain associated with the site of the subject.

Aspect 74: The device of any preceding aspect, wherein the combination of reciprocating mechanical manipulation and thermal effects transmitted produces a tissue manipulation treatment and thermal treatment effective to improve a wound associated with the site of the subject.

Aspect 75: The device of any preceding aspect, wherein the combination of reciprocating mechanical manipulation and thermal effects transmitted produces a tissue manipulation treatment and thermal treatment effective to improve blood flow to the site of the subject.

Aspect 76: The device of any preceding aspect, wherein the combination of reciprocating mechanical manipulation and thermal effects transmitted produces a tissue manipulation treatment and thermal treatment effective to enhance healing associated with the site of the subject.

Aspect 77: The device of any preceding aspect, wherein the reciprocating mechanical manipulation produces a tissue manipulation treatment effective to shear and stretch tissue at the site of the subject.

Aspect 78: The device of any preceding aspect, wherein the reciprocating mechanical manipulation produces a tissue manipulation treatment effective to provide myofascial release at the site of the subject.

Aspect 79: The device of any preceding aspect, wherein the reciprocating mechanical manipulation produces a tissue manipulation treatment effective to relieve muscle pain and tension at the site of the subject.

Aspect 80: The device of any preceding aspect, wherein the reciprocating mechanical manipulation produces a tissue manipulation treatment effective to stimulate the site of the subject.

Aspect 81: The device of any preceding aspect, wherein at least a portion of the casing is comprised of a thermally conductive and rigid material.

Aspect 82: The device of any preceding aspect, wherein the thermally conductive and rigid material comprises at least one of metal and thermally-conductive thermoplastic compositions.

Aspect 83: The device of any preceding aspect, wherein the thermal element is configured to modulate a temperature of a contacted area associated with the site of the subject.

Aspect 84: The device of any preceding aspect, wherein the reciprocating mechanical manipulation is initiated by activating the propulsion source using the switch.

Aspect 85: The device of any preceding aspect, wherein the thermal element comprises at least one of metal ingots, ice packs, low freezing point liquid packs, low freezing point gel packs, ceramics, heat sinks, Peltier coolers, thermoelectric heat pumps, and thermoelectric coolers.

Aspect 86: The device of any preceding aspect, wherein the thermal element comprises at least one of heat packs, chemical reactive thermal packs, thermal gel packs, thermal clay packs, calcium chloride-based heat packs, supersaturated sodium acetate-based heat packs, Peltier heaters, electric heating elements, metal heating elements, ceramic heating elements, polymer positive temperature coefficient (PTC) heating elements, and composite heating elements.

Aspect 87: The device of any preceding aspect, wherein the thermal element comprises at least one of Peltier coolers, thermoelectric heat pumps, thermoelectric coolers, Peltier heaters, and electric heating elements; and wherein the device is further configured to initiate at least one thermal effect by activating the thermal element using a switch or controller, or combinations thereof.

Aspect 88: The device of any preceding aspect, wherein the switch is configured to allow selective activation of the propulsion source while the head attachment is positioned on or secured against a subject.

Aspect 89: The device of any preceding aspect, wherein the switch is configured to allow selective activation of the thermal element while the head attachment is positioned on or secured against a subject.

Aspect 90: The device of any preceding aspect, wherein the switch comprises a remote switch configured to remotely activate the propulsion source or thermal element, or combinations thereof.

Aspect 91: The device of any preceding aspect, wherein the switch comprises a wireless device configured to wireless activate at least one of the propulsion source or thermal elements.

Aspect 92: The device of any preceding aspect, wherein the wireless switch comprises a wireless device configured to transmit instructions regarding operation of the device.

Aspect 93: The device of any preceding aspect, wherein the wireless device is operational to at least one of: present information regarding the operation of the device to a user via one or more graphic user interfaces on the wireless device, receive instructions regarding operation of the device from a user, and transmit the instructions to the device.

Aspect 94: The device of any preceding aspect, wherein the wireless switch comprises a wireless device having application software configured to transmit instructions regarding operation of the device.

Aspect 95: The device of any preceding aspect, wherein the application software is operational to at least one of: present information regarding operation of the device to a user via one or more graphic user interfaces on the wireless device, receive instructions regarding operation of the device from a user, and transmit the instructions to the device.

Aspect 96: The device of any preceding aspect, wherein operation of the device comprises at least one of selective activation of the propulsion source, selective activation of the thermal element, selection of a parameter associated with the propulsion device, selection of a parameter associated with the thermal effects; and selection of an activation duration.

Aspect 97: The device of any preceding aspect, wherein a thermal effect parameter comprises at least one of a thermal effect cycle and a temperature associated with at least one thermal effect.

Aspect 98: The device of any preceding aspect, wherein the casing is dimensioned to have a length of from about 3 inches to about 36 inches.

Aspect 99: The device of any preceding aspect, wherein the casing is dimensioned to have a width of from about 3 inches to about 12 inches.

Aspect 100: The device of any preceding aspect, wherein the casing is dimensioned to have a length of from about 3 inches to about 36 inches, and a width of from about 3 inches to about 12 inches; and wherein the casing is shaped to have a convex shape adapted to be secured along a curvature of a subject's back.

Aspect 101: The device of any preceding aspect, wherein the application area is constructed to transmit the thermal effects from the thermal element to the subject's skin and to transmit reciprocating mechanical energy from the propulsion source to the subject's skin,

Aspect 102: The device of any preceding aspect wherein the site comprises at least one of a site of pain and/or a site of irritation.

Aspect 103: The device of any preceding aspect wherein the site comprises at least one of a site of injury and/or a site of a wound.

Aspect 104: The device of any preceding aspect, wherein the thermal element is capable of modulating the subject's skin to a temperature of between about 15° F. (−9.4° C.) and about 120° F. (48.9° C.).

Aspect 105: The device of any preceding aspect, wherein the thermal element is capable of cooling the subject's skin to a temperature of below about 45° F. (7.2° C.).

Aspect 106: The device of any preceding aspect, wherein the thermal element is capable of cooling the subject's skin to a temperature of between about 15° F. (−9.4° C.) and about 28° F. (−2.20 C).

Aspect 107: The device of any preceding aspect, wherein the thermal element is capable of heating the subject's skin to a temperature of at least about 120° F. (48.9° C.).

Aspect 108: The device of any preceding aspect, wherein the thermal element is capable of heating the subject's skin to a temperature of between about 95° F. (35° C.) and about 120° F. (48.9° C.).

Aspect 109: The device of any preceding aspect, wherein reciprocating mechanism allows for conversion of continuous-direction rotary motion to reciprocating linear motion.

Aspect 110: The device of any preceding aspect, wherein the reciprocating mechanism comprises a DC motor driving a pinion gear, wherein the pinion gear is engaged with an involute slot gear rack, and wherein a pin attached to the pinion gear follows a guide track to thereby allow side-to-side motion.

Aspect 111: The device of any preceding aspect, wherein the propulsion source and/or reciprocating mechanism comprises at least one vertical linear guide configured to orientation of the involute slot gear rack.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

The patentable scope of the invention is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed:

1. A device for stimulating or manipulating tissue at a site in a subject, the device comprising:
a) a propulsion source capable of producing motion; and
b) a manipulating head attached to the propulsion source and comprising a casing with an application area, the application area having one or more contact portions configured to contact at least one site of a subject;
the propulsion source and the manipulating head being cooperatively structured and disposed to cause movement of the application area in response to the produced motion; and
wherein the propulsion source and application area are operatively positioned relative to one another such that the propulsion source and contact portions cooperate to manipulate tissue at the site of the subject.

2. The device of claim 1, wherein the propulsion source is adjustably mounted to a frame assembly, the frame assembly comprising a plurality of side frames connected with a central frame member.

3. The device of claim 2, wherein the frame assembly is configured to allow an attached propulsion source to travel along the central frame member.

4. The device of claim 3, wherein the propulsion source comprises a reciprocating mechanism capable of producing a reciprocating linear motion.

5. The device of claim 4, wherein the contact portions of the application area comprise integrated raised portions of the application area.

6. The device of claim 5, wherein the integrated raised portions have a bladed contact surface profile.

7. The device of claim 5, wherein proximal ends of the integrated raised portions are rounded.

8. The device of claim 4, wherein the contact portions comprise a plurality of contact members attached or disposed on the application surface.

9. The device of claim 4, further comprising a controller communicatively connected to the device and configured to control operation of one or more device components based on instructions regarding operation of the device.

10. The device of claim 9, further comprising a control unit communicatively connected to the controller and configured to transmit instructions regarding the operation of the device to the controller; wherein the control unit comprises a switch in operative communication with the device for selectively activating one or more of the device components.

11. The device of claim 10, wherein the control unit comprises a mobile device having application software configured to transmit instructions regarding the operation of the device to the controller.

12. The device of claim 4, wherein the manipulating head is pivotably connected to the propulsion source using a spring-assisted connecting mechanism.

13. The device of claim 4, wherein a housing containing the propulsion source is pivotably or rotatably connected to a device arm using a connecting mechanism; and wherein the housing containing the propulsion source is configured to rotate or pivot about an axis to a component or portion of the device to which it is connected.

14. The device of claim 4, wherein the propulsion source is configured to move the manipulating head in a predefined manner while the application area of the casing is in contact with the subject to thereby produce a tissue manipulation treatment effective to provide myofascial release or relief of muscle pain and tension at the site of the subject.

15. The device of claim 14, wherein the propulsion source comprises at least one of: a motor, screw, worm screw, barrel screw, wheel and axle, cam, rod, gear, track, belt, chain, rack and pinion, or a combination thereof.

16. The device of claim 15, wherein the reciprocating mechanism comprises a pinion gear engaged with an involute slot gear rack, and wherein a pin attached to the pinion gear follows a guide track to thereby allow side-to-side motion.

17. A system for stimulating or manipulating tissue at a site in a subject, the system comprising:
a) a device comprising:
i) a propulsion source comprising a reciprocating mechanism capable of producing a reciprocating linear motion; and
ii) a manipulating head attached to the propulsion source and comprising a casing with an application area, the application area having one or more contact portions configured to contact at least one site of a subject, the propulsion source and the manipulating head being cooperatively structured and disposed to cause movement of the application area in response to the produced motion; and the propulsion source and application area are operatively positioned relative to one another such that the propulsion source and contact portions cooperate to manipulate tissue at the site of the subject.
b) a frame assembly comprising a plurality of side frames hingedly connected to a central frame member having first and second opposed sides and a base connected to at least one end of the frame member; wherein the frame assembly is configured to detachably connect or attach with the casing;
c) a controller communicatively connected to the device and configured to control operation of the one or more device components based on instructions regarding operation of the device; and
d) a control unit communicatively connected to the controller and configured to transmit instructions regarding the operation of the device to the controller;
wherein the propulsion source is configured to move the casing in a predefined manner while the application area of the casing is in contact with the subject to thereby manipulate the tissue;
wherein the propulsion source is configured to move the manipulating head in a predefined manner while the application area of the casing is in contact with the subject to thereby manipulate the tissue.

18. The system of claim 17, wherein the reciprocating mechanism comprises a motor driving a pinion gear; wherein the pinion gear is engaged with an involute slot gear rack; and wherein a pin attached to the pinion gear follows a guide track to thereby allow side-to-side linear motion.

19. The system of claim 18, further comprising at least one thermal element capable of producing thermal effects selected from at least one of heat or cold.

20. The system of claim 18, wherein the propulsion source and thermal element are operatively positioned relative to one another such that the propulsion source and thermal element cooperate to provide fascia stimulation and thermal effects to the site of the subject.
